SEQUENCE LISTING (157 sequences)

SEQ ID NO: 1 - DNA, 1625 bp, Artificial Sequence
  pLAT14-7rci CDS
cdsacactagctgagaaatgacgtccgttaacgcaaagctcctttaccattacgtcctaaccaactttttcaacctttgcttgtttccgttaacggcgttacttgccggaaaagcctctacgcttaccacaaacgatctctaccacttctattcccatctccaacacaaccttgtaaccgtaattttactctttgctttctcctctttcggtttggttctctacgttgtaacccggcgcagaccggtttacctcgttgactactcgtgctaccttccaccaccgcatctcaaagttagtgtttctaaggtcatggatattttctaccaaataagaaaagctgatacctcacgaaacgtggcatgcgatgatccatcctcgcttgatttcctgaggaagattcaagaacgttcaggtctaggtgatgaaacctacagtcccccgggactcattcacgtgcccccacaaaagacttttgcagcttcacgtgaagagacagagcaggtaatcatcggtgcgttagaaaagttattcgagaacaccaaagttaaccctagagagattggtatacttgtggtcaactcaagcatgtttaatccaactccttcgctatctgcgatggtcgttaacactttcaagctccgaagcaacatcaaaagctttagtctcggaggaatgggttgtagtgctggtgtcatcgccattgatcttgcaaaggacttgttgcatgttcataaacacacttatgcacttgtggtgagcactgagaacatcactcaaggcatttatgctggcgaaaatagatcaatgatggttagcaattgcttgtttcgtgttggtggggcagcgattttgctatccaacaagccgggagatcggagacggtccaagtacaagttatgtcatactgttcggactcataccggagctgatgacaagtcttttcgatgtgtgcaacaaggagacgatgagagcggtaaaatcggagtttgtctgtcaaaggacataacagttgttgcggggacagcgcttaagaaaaacatagcaacgttaggtccgttgattcttcctttaagcgaaaagtttctgttcttagttaccttcatcgccaagaaacttttgaaggacaagatcaagcactattacgtcccggatttcaagcttgctattgaccatttctgtattcatgccggaggcagagccgtaatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttccgcgaatagtccttgggaacattgcatcgatagatatccggttcaaattgattctgattcatcaaaatcagagactcatgtcaaaaacggtcggacctaattcatctatctgagtgttaaccatttactgttttttatttttctttccttctttcttttattggttatgatttgataatgtttatgatgt

SEQ ID NO: 2 - protein, 505 aa, Artificial Sequence
  pLat14-7rci amino acid sequence
MTSVNAKLLYHYVLTNFFNLCLFPLTALLAGKASTLTTNDLYHFYSHLQHNLVTVILLFAFSSFGLVLYVVTRRRPVYLVDYSCYLPPPHLKVSVSKVMDIFYQIRKADTSRNVACDDPSSLDFLRKIQERSGLGDETYSPPGLIHVPPQKTFAASREETEQVIIGALEKLFENTKVNPREIGILVVNSSMFNPTPSLSAMVVNTFKLRSNIKSFSLGGMGCSAGVIAIDLAKDLLHVHKHTYALVVSTENITQGIYAGENRSMMVSNCLFRVGGAAILLSNKPGDRRRSKYKLCHTVRTHTGADDKSFRCVQQGDDESGKIGVCLSKDITVVAGTALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSDSSKSETHVKNGRT

SEQ ID NO: 3 - DNA, 1627 bp, Artificial Sequence
  pLat 14-13 CDS
cactagctgagaaatgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggtagccatttgcttgttccgtgttggtggcgcagcgattttgctctccaacaagcccggagatcggagacggtccaagtacaagttatgtcatacttttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggacctaattcatctatctgaatctgaatgttaaccatttagtgtttttttttttcctttccttctttcttttattggttaagatttgataatgtttatgatgt

SEQ ID NO: 4 - protein, 502 aa, Artificial Sequence
  pLAT14-13 amino acid
MTSVNAKLLYHYVLTNFFNLCLFPLTALLAGKASRLTSNDLYHFYSHLQHNLLTVILLFAFTAFGLVLYIVTRPKPVYLVDYSCYLPPPHLKVSVSKAMDIFYQIRKADTSRNVACDDPSSLDFLRKIQERSGLGDETYSPQGLINVPPRKTFAASREETEQVIIGALDKLFENTKVNPREIGILWNSSMFNPTPSLSAMVVNTFKLRSNIKSFSLGGMGCSAGVIAIDLAKDLLHVHKNTYALWSTENITQGIYAGENRSMMVAICLFRVGGAAILLSNKPGDRRRSKYKLCHTFRTHTGADDMSFRCVQQGDDESGKIGVCLSKDITWAGIALKKNIATLGPLLLPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSGSSKSDTHVKNGRT

SEQ ID NO: 5 - DNA, 1152 bp, Artificial Sequence
  AtFad2-At3g12120 CDS
atgggtgcaggtggaagaatgccggttcctacttcttccaagaaatcggaaaccgacaccacaaagcgtgtgccgtgcgagaaaccgcctttctcggtgggagatctgaagaaagcaatcccgccgcattgtttcaaacgctcaatccctcgctctttctcctaccttatcagtgacatcattatagcctcatgcttctactacgtcgccaccaattacttctctctcctccctcagcctctctcttacttggcttggccactctattgggcctgtcaaggctgtgtcctaactggtatctgggtcatagcccacgaatgcggtcaccacgcattcagcgactaccaatggctggatgacacagttggtcttatcttccattccttcctcctcgtcccttacttctcctggaagtatagtcatcgccgtcaccattccaacactggatccctcgaaagagatgaagtatttgtcccaaagcagaaatcagcaatcaagtggtacgggaaatacctcaacaaccctcttggacgcatcatgatgttaaccgtccagtttgtcctcgggtggcccttgtacttagcctttaacgtctctggcagaccgtatgacgggttcgcttgccatttcttccccaacgctcccatctacaatgaccgagaacgcctccagatatacctctctgatgcgggtattctagccgtctgttttggtctttaccgttacgctgctgcacaagggatggcctcgatgatctgcctctacggagtaccgcttctgatagtgaatgcgttcctcgtcttgatcacttacttgcagcacactcatccctcgttgcctcactacgattcatcagagtgggactggctcaggggagctttggctaccgtagacagagactacggaatcttgaacaaggtgttccacaacattacagacacacacgtggctcatcacctgttctcgacaatgccgcattataacgcaatggaagctacaaaggcgataaagccaattctgggagactattaccagttcgatggaacaccgtggtatgtagcgatgtatagggaggcaaaggagtgtatctatgtagaaccggacagggaaggtgacaagaaaggtgtgtactggtacaacaataagttatga

SEQ ID NO: 6 - protein, 383 aa, Artificial Sequence
  AtFad2-At3g12120 protein
MGAGGRMPVPTSSKKSETDTTKRVPCEKPPFSVGDLKKAIPPHCFKRSIPRSFSYLISDIIIASCFYYVATNYFSLLPQPLSYLAWPLYWACQGCVLTGIWVIAHECGHHAFSDYQWLDDTVGLIFHSFLLVPYFSWKYSHRRHHSNTGSLERDEVFVPKQKSAIKWYGKYLNNPLGRIMMLTVQFVLGWPLYLAFNVSGRPYDGFACHFFPNAPIYNDRERLQIYLSDAGILAVCFGLYRYAAAQGMASMICLYGVPLLIVNAFLVLITYLQHTHPSLPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 7 - DNA, 1161 bp, Artificial Sequence
  AtFad3-At2g29980 CDS
atggttgttgctatggaccaacgcaccaatgtgaacggagatcccggcgccggagaccggaagaaagaagaaaggtttgatccgagtgcacaaccaccgttcaagatcggagatataagggcggcgattcctaagcactgttgggttaagagtcctttgagatcaatgagttacgtcgtcagagacattatcgccgtcgcggctttggccatcgctgccgtgtatgttgatagctggttcctttggcctctttattgggccgcccaaggaacacttttctgggccatctttgttctcggccacgactgtggacatgggagtttctcagacattcctctactgaatagtgtggttggtcacattcttcattctttcatcctcgttccttaccatggttggagaataagccaccggacacaccaccagaaccatggccatgttgaaaacgacgagtcatgggttccgttaccagaaagggtgtacaagaaattgccccacagtactcggatgctcagatacactgtccctctccccatgctcgcatatcctctctatttgtgctacagaagtcctggaaaagaaggatcacattttaacccatacagtagtttatttgctccaagcgagagaaagcttattgcaacttcaactacttgttggtccataatgttcgtcagtcttatcgctctatctttcgtcttcggtccactcgcggttcttaaagtctacggtgtaccgtacattatctttgtgatgtggttggatgctgtcacgtatttgcatcatcatggtcacgatgagaagttgccttggtatagaggcaaggaatggagttatctacgtggaggattaacaacaattgatagagattacggaatctttaacaacattcatcacgacattggaactcacgtgatccatcatctcttcccacaaatccctcactatcacttggtcgacgccacgaaagcagctaaacatgtgttgggaagatactacagagaaccaaagacgtcaggagcaataccgatccacttggtggagagtttggtcgcaagtattaagaaagatcattacgtcagcgacactggtgatattgtcttctacgagacagatccagatctctacgtttacgcttctgacaaatctaaaatcaattaa

SEQ ID NO: 8 - protein, 386 aa, Artificial Sequence
  atFad3-At2g29980 protein
MVVAMDQRTNVNGDPGAGDRKKEERFDPSAQPPFKIGDLRAAIPKHCWVKSPLRSMSYVVRDIIAVAALAIAAVYVDSWFLWPLYWAAQGTLFWAIFVLGHDCGHGSFSDIPLLNSVVGHILHSFILVPYHGWRISHRTHHQNHGHVENDESWVPLPERVYKKLPHSTRMLRYTVPLPMLAYPLYLCYRSPGKEGSHFNPYSSLFAPSERKLIATSTTCWSIMFVSLIALSFVFGPLAVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPKTSGAIPIHLVESLVASIKKDHYVSDTGDIVFYETDPDLYVYASDKSKIN

SEQ ID NO: 9 - DNA, 1521 bp, Artificial Sequence
  AtFael-At4g34520 CDS
atgacgtccgttaacgttaagctcctttaccgttacgtcttaaccaactttttcaacctctgtttgttcccgttaacggcgttcctcgccggaaaagcctctcggcttaccataaacgatctccacaacttcctttcctatctccaacacaaccttataacagtaactttactctttgctttcactgttttcggtttggttctctacatcgtaacccgacccaatccggtttatctcgttgactactcgtgttaccttccaccaccgcatctcaaagttagtgtctctaaagtcatggatattttctaccaaataagaaaagctgatacttcttcacggaacgtggcatgtgatgatccgtcctcgctcgatttcctgaggaagattcaagagcgttcaggtctaggtgatgagacgtacagtcctgagggactcattcacgtaccaccgcggaagacttttgcagcgtcacgtgaagagacagagaaggttatcatcggtgcgctcgaaaatctattcgagaacaccaaagttaaccctagagagattggtatacttgtggtgaactcaagcatgtttaatccaactccttcgctatccgctatggtcgttaatactttcaagctccgaagcaacatcaaaagctttaatctaggaggaatgggttgtagtgctggtgttattgccattgatttggctaaagacttgttgcatgttcataaaaacacttatgctcttgtggtgagcactgagaacatcacacaaggcatttatgctggagaaaatagatcaatgatggttagcaattgcttgtttcgtgttggtggggccgcgattttgctctctaacaagtcgggagaccggagacggtccaagtacaagctagttcacacggtccgaacgcatactggagctgatgacaagtcttttcgatgtgtgcaacaagaagacgatgagagcggcaaaatcggagtttgtctgtcaaaggacataaccaatgttgcggggacaacacttacgaaaaatatagcaacattgggtccgttgattcttcctttaagcgaaaagtttctttttttcgctaccttcgtcgccaagaaacttctaaaggataaaatcaagcattactatgttccggatttcaagcttgctgttgaccatttctgtattcatgccggaggcagagccgtgatcgatgagctagagaagaacttaggactatcgccgatcgatgtggaggcatctagatcaacgttacatagatttgggaatacttcatctagctcaatttggtatgaattagcatacatagaggcaaagggaagaatgaagaaagggaataaagcttggcagattgctttaggatcagggtttaagtgtaatagtgcggtttgggtggctctacgcaatgtcaaggcatcggcaaatagtccttggcaacattgcatcgatagatatccggttaaaattgattctgatttgtcaaagtcaaagactcatgtccaaaacggtcggtcctaa

SEQ ID NO: 10 - protein, 506 aa, Artificial Sequence
  AtFaeI-At4g34520 protein
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 11 - DNA, 1155 bp, Artificial Sequence
  pLAT12-1 CDS
atgggtgcaggtggaagaatgccagttccttcttcttcttccaagaaatctgaaaccgatgccataaagcgtgtgccctgcgagaaaccaccgttcacgctgggagatctgaagaaagcaatcccaccgcagtgtttcaaacgctctatccctcgctctttctcctaccttatcactgacatcattattgcctcctgcttctactacgtcgccaccaattacttttctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacaggatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgcgaacgcctccagatatatctctctgatgccggtattctagcagtctgttttgggctttaccgttacgccgctgcacaaggattggcctcgatgatctgcctctacggagtaccacttctgatagtgaacgcgttcctcgtcttgatcacttacttgcagcacactcatcctgcgttgcctcactacgattcatccgagtgggattggcttaggggagctttggctaccgtagacagagactatggaatcttgaataaggtgttccacaacatcacggacacacatgtggctcatcatctgttctcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatga

SEQ ID NO: 12 - protein, 384 aa, Artificial Sequence
  pLAT12-1 amino acid
MGAGGRMPVPSSSSKKSETDAIKRVPCEKPPFTLGDLKKAIPPQCFKRSIPRSFSYLITDIIIASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPAGRIMMLTVQFVLGWPLYLAFNVSGRPYDGFACHFFPNAPIYNDRERLQIYLSDAGILAVCFGLYRYAAAQGLASMICLYGVPLLLVNAFLVLLTYLQHTHPALPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 13 - DNA, 879 bp, Artificial Sequence
  pLAT-12-4 CDS
atgggtgcaggtggaagaatgccagttccttcttcttcttcttccaagaaatctgaaaccgatgccataaagcgtgtgccctgcgagaaaccgccgttcacactgggagaattgaagaaagcgatcccaccgcagtgtttcaaacgctctatccctcgctctttctcctaccttatcactgacatcattgttgcctcctgcttctactacgtcgccaccaattacttctctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacaggatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgtgaacgcctccagatatatctctctgatgccggtattctagcagtctgttttgggcttttaccgttacgccgctgcacaaggattggcctcgatgatctgcctctacggagtaccacttctgatagtgaacgcgttcctcgtcttgatcacttacttgcagcacactcatcctgcgttgcctcactacgattcatccgagtgggattggcttag

SEQ ID NO: 14 - protein, 292 aa, Artificial Sequence
  pLAT12-4 amino acid
MGAGGRMPVPSSSSSKKSETDAIKRVPCEKPPFTLGELKKAIPPQCFKRSIPRSFSYLITDIIVASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPAGRIMMLTVQFVLGWPLYLAFNVSGRPYDGFACHFFPNAPIYNDRERLQIYLSDAGILAVCFGLLPLRRCTRIGLDDLPLRSTTSDSERVPRLDHLLAAHSSCVASLRFIRVGLA

SEQ ID NO: 15 - DNA, 576 bp, Artificial Sequence
  pLAT12-11 CDS
atgggtgcaggtggaagaatgccagttccttcttcttcttcttccaagaaatctgaaaccgatgccataaagcgtgtgccctgcgagaaaccgccgttcacgcttggagaactgaagaaagcaatccccccgcagtgtttcaaacgctctatccctcgctctttctcctaccttatcactgacatcattattgcctcctgcttctactacgtcgccaccaattacttctctctcctccctcagcctctctcttacttggcttggcccctctattgggcttgtcaaggctgtgtcctaaccggtgtctgggtcatagcccacgaatgcggtcaccacgcattcagcgactaccaatggcttgatgacacagttggtcttatcttccattccttccttctcgtcccttacttctcctggaagtacagtcatcgccgtcaccattccaacacaggatctctcgaaagagatgaagtatttgtcccaaagcagaagtccgctatcaagtggtatggcaaatacctcaacaaccctgctggacgcatcatgatgttgaccgtccagtttgtcctcgggtga

SEQ ID NO: 16 - protein, 191 aa, Artificial Sequence
  pLAT12-11 amino acid
MGAGGRMPVPSSSSSKKSETDAIKRVPCEKPPFTLGELKKAIPPQCFKRSIPRSFSYLLTDILIASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPAGRIMMLTVQFVLG

SEQ ID NO: 17 - DNA, 1155 bp, Artificial Sequence
  pLAT12-12 CDS
atgggtgcaggtggaagaatgccggttccttcttcttcttccaagaaatcagaaaccgatgccataaagcgtgtgccctgcgagaaaccaccgttcacgctgggagatctgaagaaagcaatcccaccgcagtgtttcaaacgctctatccctcgctctttctcctaccttatcactgacatcattattgcctcctgcttctactacgtcgccaccaattacttttctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacaggatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgtgaacgcctccagatatatctctctgatgccggtatcctagcagtctgttttgggctttaccgttacgttgctgcacaaggaatggcctcgatgatctgcctctacggagtaccacttctgatagtaaacgcgttcctcgtcttgatcacttacttgcagcacactcatcctgcgttgcctcactacgattcatccgagtgggattggcttaggggagctttggctaccgtagacagagactatggaatcttgaataaggtgttccacaacatcacggacacacatgtggctcatcatctgttctcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatga

SEQ ID NO: 18 - protein, 384 aa, Artificial Sequence
  pLAT12-12 amino acid
MGAGGRMPVPSSSSKKSETDAIKRVPCEKPPFTLGDLKKAIPPQCFKRSIPRSFSYLITDIIIASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPAGRIMMLTVQFVLGWPLYLAFNVSGRPYDGFACHFFPNAPIYNDRERLQIYLSDAGILAVCFGLYRYVAAQGMASMICLYGVPLLLVNAFLVLLTYLQHTHPALPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 19 - DNA, 693 bp, Artificial Sequence
  pLAT12-13 CDS
atgggtgcaggtggaagaatgccagttccttcttcttcttcttccaagaaatctgaaaccgatgccataaagcgtgtgccctgcgagaaaccgccgttcacgcttggagaactgaagaaagcaatccccccgcagtgtttcaaacgctctatccctcgctctttctcctaccttatcactgacatcattgttgcctcctgcttctactacgtcgccaccaattacttctctctcctccctcagcctctctcttacttggcttggcctctctactgggcctgtcaaggctgtgtcctaaccggtgtctgggtcatagctcacgaatgcggtcaccacgcattcagcgactaccaatggcttgatgacacagttggtcttatcttccattccttccttctcgtcccttacttctcctggaagtacagtcatcgtcgtcaccattccaacacaggatctctcgaaagagatgaagtatttgtcccaaagcagaaatcagctatcaagtggtatggcaaatacctcaacaaccctcctggacgcatcatgatgttaaccgtccagtttgtcctcgggtggcccttgtacttggcctttaacgtctcgggcagaccgtacgacgggttcgcttgccatttcttccccaacgctcccatctacaacgaccgcgaacgcctcccagatatatctctctga

SEQ ID NO: 20 - protein, 230 aa, Artificial Sequence
  pLAT12-13 amino acid
MGAGGRMPVPSSSSSKKSETDAIKRVPCEKPPFTLGELKKAIPPQCFKRSIPRSFSYLLTDILVASCFYYVATNYFSLLPQPLSYLAWPLYWACQGCVLTGVWVIAHECGHHAFSDYQWLDDTVGLLFHSFLLVPYFSWKYSHRRHHSNTGSLERDEVFVPKQKSAIKWYGKYLNNPPGRIMMLTVQFVLGWPLYLAFNVSGRPYDGFACHFFPNAPIYNDRERLPDISL

SEQ ID NO: 21 - DNA, 1164 bp, Artificial Sequence
  pLAT13-28 CDS
atggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtccgagacatttgcgccgtcgcggctcttgccgtcgccgccgtgtattttaacagctggttcctctggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacacaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctatcattcatcttcggtccactctccgttcttaaagtctatggtgtgccgtacatcatctttgtgatgtggttggacgctgtcacttatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccactaaagcagctaaacatgtgttgggaagatactacagagaaccacaaacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 22 - protein, 384 aa, Artificial Sequence
  pLAT13-28 amino acid
MWAMDKRSNVNGDPGAGDRNKKGEGFDPSAQPPFKIGDIRAAIPKHCWVKSPLRSMSYWRDICAVAALAVAAVYFNSWFLWPLYWAAQGTLFWAIFVLGHDCGHGSFSDIPLLNSWGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQTSGAIPIHLVESLVASIKKDHYVSDTGDIVFYETDPDLYVYASDKSKIN

SEQ ID NO: 23 - DNA, 1164 bp, Artificial Sequence
  pLAT13-29 CDS
atggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtccgagacatttgcgccgtcgctgctcttgccgtcgccgccgtgtattttaacagctggttcctttggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacgcaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctgtcattcatcttcggtccactttccgttcttaaagtctacggtgtgccgtacatcatctttgtgatgtggttggacgctgtcacgtatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccacaaaatcagctaaacatgtgttgggaagatactacagagaaccacagacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 24 - protein, 387 aa, Artificial Sequence
  pLAT13-29 amino acid
MVVAMDKRSNVNGDPGAGDRNKKGEGFDPSAQPPFKLGDLRAAIPKHCWVKSPLRSMSYVVRDICAVAALAVAAVYFNSWFLWPLYWAAQGTLFWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKSAKHVLGRYYREPQTSGAIPIHLVESLVASIKKDHYVSDTGDIVFYETDPDLYVYASDKSKIN

SEQ ID NO: 25 - DNA, 1164 bp, Artificial Sequence
  pLAT13-30 CDS
atggttgttgctatggacaaacgtagcaatgtgaacggagatcccggcgccggagaccagaagaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacacaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctatcattcatcttcggtccactctccgttcttaaagtctatggtgtgccgtacatcatctttgtgatgtggttggacgctgtcacgtatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccacaaaagcagctaaacatgtgttgggaagatactacagagaaccacaaacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 26 - protein, 386 aa, Artificial Sequence
  pLAT13-30 aa
MVVAMDKRSNVNGDPGAGDQKKKGEGFDPSAQPPFKIGDIRAAIPKHCWVKSPLRSMSYVVRDICAVAALAVAAVYFNSWFLWPLYWAAQGTLFWAIFVLGHDCGHGSFSDIPLLNSWGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQTSGAIPIHLVESLVASIKKDHYVSDTGDIVFYETDPDLYVYASDKSKIN

SEQ ID NO: 27 - DNA, 1164 bp, Artificial Sequence
  pLAT13-32 CDS
atggttgttgctatggacaaacgtagcaatgtgaacggagatcccggcgccggagaccagaagaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacacaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctatcattcatcttcggtccactctccgttcttaaagtctatggtgtgccgtacatcatctttgtgatgtggttggacgctgtcacttatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccactaaagcagctaaacatgtgttgggaagatactacagagaaccacaaacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 28 - protein, 385 aa, Artificial Sequence
  pLAT13-32 amino acid
MVVAMDKRSNVNGDPGAGDQKKKGEGFDPSAQPPFKIGDIRAAIPKHCWVKSPLRSMSYWRDICAVAALAVAAVYFNSWFLWPLYWAAQGTLFWAIFVLGHDCGHGSFSDIPLLNSWGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQTSGAIPIHLVESLVASIKKDHYVSDTGDIVFYETDPDLYVYASDKSKIN

SEQ ID NO: 29 - DNA, 1164 bp, Artificial Sequence
  pLAT13-39 CDS
atggttgttgctatggacaaacgtagcaatgtgaacggagatcccggcgccggagaccagaagaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacacaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctgtcattcatcttcggtccactttccgttcttaaagtctacggtgtgccgtacatcatctttgtgatgtggttggacgctgtcacgtatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccacaaaatcagctaaacatgtgttgggaagatactacagagaaccacagacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 30 - protein, 386 aa, Artificial Sequence
  pLAT13-39 amino acid
MVVAMDKRSNVNGDPGAGDQKKKGEGFDPSAQPPFKIGDIRAAIPKHCWVKSPLRSMSYVVRDICAVAALAVAAVYFNSWFLWPLYWAAQGTLFWAIFVLGHDCGHGSFSDIPLLNSWGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKSAKHVLGRYYREPQTSGAIPIHLVESLVASIKKDHYVSDTGDIVFYETDPDLYVYASDKSKIN

SEQ ID NO: 31 - DNA, 1161 bp, Artificial Sequence
  pLAT13-40 CDS
atggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtccgagacatttgcgccgtcgcggctcttgccgtcgccgccgtgtattttaacagctggttctggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacacaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctatcattcatcttcggtccactctccgttcttaaagtctatggtgtgccgtacatcatctttgtgatgtggttggacgctgtcacttatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccactaaagcagctaaacatgtgttgggaagatactacagagaaccacaaacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 32 - protein, 386 aa, Artificial Sequence
  pLAT13-40 amino acid
MVVAMDKRSNVNGDPGAGDRNKKGEGFDPSAQPPFKLGDLRAAIPKHCWVKSPLRSMSYVVRDICAVAALAVAAVYFNSWFWPLYWAAQGTLFWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQTSGAIPIHLVESLVASIKKDHYVSDTGDIVFYETDPDLYVYASDKSKIN

SEQ ID NO: 33 - DNA, 1164 bp, Artificial Sequence; identical to SEQ ID NO: 21
  pLAT13-41 CDS

SEQ ID NO: 34 - protein, 387 aa, Artificial Sequence
  pLAT13-41 aa
MVVAMDKRSNVNGDPGAGDRNKKGEGFDPSAQPPFKLGDLRAAIPKHCWVKSPLRSMSYVVRDICAVAALAVAAVYFNSWFLWPLYWAAQGTLFWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQTSGAIPIHLVESLVASIKKDHYVSDTGDIVFYETDPDLYVYASDKSKIN

SEQ ID NO: 35 - DNA, 1164 bp, Artificial Sequence
  pLAT13-42rc CDS
atggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcacccttttctgggccatcttcgtcctcggccacgactgtggacatgggagtttctcagacattcctctgctgaatagtgtggttggtcacattcttcattccttcatcctcgttccttaccatggttggagaataagccaccggacgcaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctatcattcatcttcggtccactctccgttcttaaagtctatggtgtgccgtacatcatctttgtgatgtggttggacgctgtcacgtatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccacaaaagcagctaaacatgtgttggaaagatactacagagaaccacaaacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 36 - protein, 387 aa, Artificial Sequence
  pLAT13-42rc amino acid
MVVAMDKRSNVNGDPGAGDRNKKGEGFDPSAQPPFKIGDLRAAIPKHCWVKSPLRSMSYVVRDICAVAALAVAAVYFNSWFLWPLYWAAQGTLFWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLERYYREPQTSGAIPIHLVESLVASIKKDHYVSDTGDIVFYETDPDLYVYASDKSKIN

SEQ ID NO: 37 - DNA, 1627 bp, Artificial Sequence
  pLAT14-4rci CDS
acactagctgagaaatgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaagggcatttatgctggcgaaaatagatccatgatgggttagcaattgcttgttccgtgtkgtggcgcagcgatttgctctccaacaagccaggatatcggagacggtcccaagtacaagttatgtcatactgtcgacgcataccgagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggacctaattcatctatctgaatctgaatgttaaccatttagtgtttttttttttcctttccttctttcttttattggttaagatttgataatgtttatgatgta

SEQ ID NO: 38 - protein, 503 aa, Artificial Sequence
  pLAT14-4rci amino acid
MTSVNAKLLYHYVLTNFFNLCLFPLTALLAGKASRLTSNDLYHFYSHLQHNLLTVILLFAFTAFGLVLYIVTRPKPVYLVDYSCYLPPPHLKVSVSKAMDIFYQIRKADTSRNVACDDPSSLDFLRKIQERSGLGDETYSPQGLINVPPRKTFAASREETEQVIIGALDKLFENTKVNPREIGILWNSSMFNPTPSLSAMVVNTFKLRSNIKSFSLGGMGCSAGVIAIDLAKDLLHVHKNTYALWSTENITQGIYAGENRSMMVSNCLFRVGGAAILLSNKPGYRRRSKYKLCHTVRTHTGADDMSFRCVQQGDDESGKIGVCLSKDITVVAGIALKKNIATLGPLLLPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSGSSKSDTHVKNGRT

SEQ ID NO: 39 - DNA, 1631 bp, Artificial Sequence
  pLAT14-5rci CDS
acactagctgagaaatgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaatttgcttgttccgtgttggtggcgcagcgattttgcttctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtttagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggacctaattcatctatctgaatctgaatgttaaccatttagtgtttttttttttcctttccttctttcttttattggttaagatttgataatgtttatgatgta

SEQ ID NO: 40 - protein, 503 aa, Artificial Sequence
  pLAT14-5rci amino acid
MTSVNAKLLYHYVLTNFFNLCLFPLTALLAGKASRLTSNDLYHFYSHLQHNLLTVILLFAFTAFGLVLYIVTRPKPVYLVDYSCYLPPPHLKVSVSKAMDIFYQIRKADTSRNVACDDPSSLDFLRKIQERSGLGDETYSPQGLINVPPRKTFAASREETEQVIIGALDKLFENTKVNPREIGILWNSSMFNPTPSLSAMVVNTFKLRSNIKSFSLGGMGCSAGVIAIDLAKDLLHVHKNTYALWSTENITQGIYAGENRSMMVSNCLFRVGGAAILLSNKPGDRRRSKYKLCHTVRTHTGADDMSFRCVQQGDDESGKIGVCLSKDITVVAGIALKKNIATLGPLLLPLSEKFLFLVTFIAKKLLKDKIKHYYVPDFKLAIDHFCIHAGGRAVIDVLEKSLGLSPIDVEASRSTLHRFGNTSFSSIWYELAYIEAKGRMKKGNRAWQIALGSGFKCNSAVWVALCNVKASANSPWEHCIDRYPVQIDSGSSKSDTHVKNGRT

SEQ ID NO: 41 - DNA, 21 bp, Artificial Sequence
  fad2a amiRNA
taagggacgaggaggaaggaa

SEQ ID NO: 42 - DNA, 21 bp, Artificial Sequence
  fad2a 159 Star sequence
ttccttcctcccagtcccttt

SEQ ID NO: 43 - DNA, 21 bp, Artificial Sequence
  fad2a 396b Star sequence
ttcattcctcctcgtccctta

SEQ ID NO: 44 - DNA, 21 bp, Artificial Sequence
  fad2b amiRNA
tttcttgtcaccttccctgtc

SEQ ID NO: 45 - DNA, 21 bp, Artificial Sequence
  fad2b 159 Star sequence
gacagggaaggacacaagaat

SEQ ID NO: 46 - DNA, 21 bp, Artificial Sequence
  fad2b 396b Star sequence
gactgggaaggtgacaagaaa

SEQ ID NO: 47 - DNA, 21 bp, Artificial Sequence
  fad2c amiRNA
tggggaagaaatggcaagcga

SEQ ID NO: 48 - DNA, 21 bp, Artificial Sequence
  fad2c 159 Star sequence
tcgcttgccataacttcccct

SEQ ID NO: 49 - DNA, 21 bp, Artificial Sequence
  fad2c 396b Star sequence
tcgattgccatttcttcccca

SEQ ID NO: 50 - DNA, 21 bp, Artificial Sequence
  fad3a amiRNA
ttgttgttaatcctccacgta

SEQ ID NO: 51 - DNA, 21 bp, Artificial Sequence
  fad3a 159 Star sequence
tacgtggaggaacaacaacat

SEQ ID NO: 52 - DNA, 21 bp, Artificial Sequence
  fad3a 396b Star sequence
tacctggaggattaacaacaa

SEQ ID NO: 53 - DNA, 21 bp, Artificial Sequence
  fad3b amiRNA
tttcttaatacttgcgaccaa

SEQ ID NO: 54 - DNA, 21 bp, Artificial Sequence
  fad3b 159 Star sequence
ttggtcgcaagatttaagaat

SEQ ID NO: 55 - DNA, 21 bp, Artificial Sequence
  fad3b 396b Star sequence
ttgctcgcaagtattaagaaa

SEQ ID NO: 56 - DNA, 21 bp, Artificial Sequence
  feaIa amiRNA
tccctttcttcattcttccct

SEQ ID NO: 57 - DNA, 21 bp, Artificial Sequence
  faeIa 159 Star sequence
agggaagaatgtcgaaagggt

SEQ ID NO: 58 - DNA, 21 bp, Artificial Sequence
  faeIa 396b Star sequence
aggcaagaatgaagaaaggga

SEQ ID NO: 59 - DNA, 21 bp, Artificial Sequence
  faeIb amiRNA
tacacttaaaccctgatccta

SEQ ID NO: 60 - DNA, 21 bp, Artificial Sequence
  faeIb 159 Star sequence
aagacgatgagtccggcaaat

SEQ ID NO: 61 - DNA, 21 bp, Artificial Sequence
  faeIb 396b Star sequence
tagcatcagggtttaagtgta

SEQ ID NO: 62 - DNA, 21 bp, Artificial Sequence
  faeIc amiRNA
ttttgccgctctcatcgtctt

SEQ ID NO: 63 - DNA, 21 bp, Artificial Sequence
  faeIc 159 Star sequence
aagacgatgagtccggcaaat

SEQ ID NO: 64 - DNA, 21 bp, Artificial Sequence
  faeIc 396b Star sequence
aagtcgatgagagcggcaaaa

SEQ ID NO: 65 - DNA, 11042 bp, Artificial Sequence
  GM-159-KS332
ggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatcctcgaagagaagggttaataacacacttttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggcccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgggccgctctagaactagtggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagtgtttaaacggagctccttgaagtccaattgaggatcttactgggtgaattgagctgcttagctatggatcccacagttctacccatcaataagtgcttttgtggtagtcttgtggcttccatatctggggagcttcatttgcctttatagtattaaccttctttggattgaagggagctctagtttaaaccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 66 - DNA, 10651 bp, Artificial Sequence
  GM-396b-KS332
ggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatcctcgaagagaagggttaataacacacttttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggcccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgggccgctctagaactagtggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctgtttaaaccacagctttcttgaacttcttatgcatcttatatctctccacctccaggattttaagccctagaagctcaagaaagctgtgggagtttaaactatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacatannnatctcttaatctagagattttatggctttgttatatataaggc

SEQ ID NO: 67 - DNA, 7640 bp, Artificial Sequence
  pKR2007
gatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctgtttaaaccacagctttcttgaacttcttatgcatcttatatctctccacctccaggattttaagccctagaagctcaagaaagctgtgggagtttaaactatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacatannnatctcttaatctagagattttatggctttgttatatataaggcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttg

SEQ ID NO: 68 - DNA, 8031 bp, Artificial Sequence
  pKR2009
agcttggatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagtgtttaaacggagctccttgaagtccaattgaggatcttactgggtgaattgagctgcttagctatggatcccacagttctacccatcaataagtgcttttgtggtagtcttgtggcttccatatctggggagcttcatttgcctttatagtattaaccttctttggattgaagggagctctagtttaaaccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaaca

SEQ ID NO: 69 - DNA, 5288 bp, Artificial Sequence
  pLF305
ccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactgga

SEQ ID NO: 70 - DNA, 4137 bp, Artificial Sequence
  pLF306
aaaagatgaaaaaagtataaaaggtgtaatgagagaaaaattaaataaatatgagtaatttgttgttttaaaataaatgtatttttgatatttttgtttatttaaagtaaatgatgagtggaaatgtatgatgttaaaagatatgatataaatagtgaattggaggttttatataagaaagttaatattagtaagtattaaagaatttaagatatatggggtttagtagtagggtttgggtagtgagtgtaataaagttgaaagtttttggttaggttagttttgattattattatagttaaagggaaaattaattaaaggggattatgaaggaagggaaggaaaagaattgaggatttatgggtgaattgagtgttagtatggataagtttaataataagtgttttgtggtagtttgtggttatattggggagttatttgtttatagtattaatttttttgtatttgtatttttttttttataataatttaaatttgttatagattaaatttaaatgttttttttgaagtttttgttttttttttgatgatgttttgtgtggagtaattgtgaggtatgtgatgattagatatattaatttgtgtgatagaaggtgaggttgaaatgaatttgttttttgattttaggaaagttttttgttgagtaataattttaattagttttaattgaatattattttattgtattttgttagttttattgttgaattgagtgatattttaggaaatttttataaattttttataagattatgtttggtttgttatttggttgtgttggttatttgttaatatttattgatggtgatgagaatgtaaattttgggttatttgttggttaataaaaggagtgttattgaaagataagaggagattggttttatataaataaataaagatgaatattagtgtgttgatgttttaggataatttttgtttgaaataatatggtaatgtttgttaaatttgtgtaataattttatgattttttggattgttggatttttataaaaaattggggaagtatgaataaaatgatgtaggtgtaagagtatggagagatggaatattgtatgaatgtaaagtataataatgagtattatttttatgaataaaaaaggatgttatgatatattaaattattatgattattgtttatgataaattttttattattataaatattgaatgtgaggttatggaatgttaaatagtaaaaaaaaatgtgtatataagattttaaaaatttaattagattgtgaagagaataagtgttaagaagaataaaattataatggaagaagtttgttatttatatattatatattaattatgtattatattaggatgttaaggagaataaaattataaagagagaagtttgtatatttatatattatatataatttatatattatattatattatttaatgttttataaggtttgatatgatattttaatattttagttgatatgtatatgaaagggtatatttgaattttattgtataaaggttggatatttaaagtgggttatttaattttattgttttaagataaaaaaaaaattatgagttggtttgataaaatattgaaggatttaaaataataataaataaatataatatatgtatataaatttattataatataaatttattataaaaaagtaaatattgtataaattataaatgtttagttgtggagaattaattatttaaagagagtaaaatatttgatttttggttatttaaaaattattatttaaatatatgaaatttttttttttatagaaagaataaaattaaattaagaaggaaatggtgtaatttataaaaattaaagaaagtaagtagagaaaaaaaaaaaagaaaggaaattttttaatttgagttgtttgtttgtgataatttatgagtaaaaataaataattttagagtagagaatggttgagtgtgttagttttttattttatttttttatagaaagaataaataaaataaaatgagaattagggatgtttaaaagttggatgtgagggggtttatagtgtataaatgtatgtgtatgataataaggttatgtattaattgtagggtttaagaaatatgtatatggtgattagtaaattgttgatggatagttaagaggaagaaagtgaggtgagggttgttgtggatgttaagaaagtgtgagttgggagtgatgtgtagaggttttagtatagaaagggagagaaagggtgtgatagtatttttataggttaatgtatgaaaaatttaagtgagttttgttatgaggtagagtagaaaagataaaggatttttgagatttttttttgggtaattgtgttgaaaaaaaaaaagtaagggtggtttgtttgggataagagtaaattttttgaaggtaatggttagagaggagataaaatatgttttagtgtaggtagttaggaattaagaattgtagagtaatatgttgtaattgttaagtggtgtgagtgggataagtgtgtttagggttggataagagatagttaggataagggagggtgggtgaaggggggttgtgaaagagttggaggaagataagaatgagatataaggtgagattgagaaaggagttgaagggagaaaggggaaggtatggtaagggagggtggaaaggagaggagagggagttagggggaaagtggtattttatagttgtgggtttgattgattgaggtgatttttgtgatgtgtaggggggggagtatggaaaaagagaagggtttttaggtttggttttgtggttttgtaatgttttttggttattgatttgtggataagtattagtttgagtgagtgatagtggaggaagagaggaggagtagtgaggaggaagggaagaggaatagaaagttgggttgggattattaatgaggttgatagattgatggaaattaatagatatatagggagaaaaggtttttagaaataattttgtttaatttaagaaggagatataatggaaaagtgaataggagttgtgagaagttttgatgaaaagttgaaggttgatgatgagttggaggggaagaattgtgtttagttgatgtaggaggggtggatatgttggggtaaatagtgggatggttttaaaagatgttatgtttatggatttgatggggtgattggaagtgttgaattggggaattaggagagtgatattgattggtgaagggtgtagttgaagatgtgaaagaatggtgtttgagggtgggaggtatggatggatgtggggatttagagagaggggttggattggagaaggaatggtaataataatgggtgatttatatgggattgtgatatgtgtatatggaaatgtgatggagaagtagtggtgtggaggttgatgagtgatgtttggggaggatggaagtggatgtgagggatttggtaaaatgttgaggaaatgggataaagggtattgatggaggagggatgttggggattaatagaggtgaaattttttggagggtggttggttgtatggagagagaggtattgagggaggatggagttgaggatggggtggggtatatgtgattggtttgaaattatagagttggttgaggaatttgatgatgagttggggagggtgatggagaatgtgatggaggggatgtggggtaaaaatggagaagggggttggagatggtgtgtagaagtatggatagtggaaagagagatgtgagggaaaggaatagtgaggtaagttggatgatggtgtaaaaaggaaaggaagtgagttggtgtgagtgagaataatagataattggggttaaagggtttgaggggttttttgtgaaaggaggaatatatggatgatgggggaagttttgatatgttatttggttattaattaatttggtaaagtgtataatagttattatttatataattaatttggtagttgaatgaaaatgatagttttggatataagaaaaagaaggaaaaaagaagaaaaaaaatgagagtattttgatagaatgttaagttataaaattaaaaaaaagaataaaagtggaatattatatagtgataggatgggtaagaaaaaaaaatgga

SEQ ID NO: 71 - DNA, 5288 bp, Artificial Sequence
  pLF307
ccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagtcgcttgccataacttccccttgaggatcttactgggtgaattgagctgcttagctatggatcccacagttctacccatcaataagtgcttttgtggtagtcttgtggcttccatatctggggagcttcatttgcctttatagtattaaccttctggggaagaaatggcaagcgacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactgga

SEQ ID NO: 72 - DNA, 5288 bp, Artificial Sequence
  pLF308
ccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagtacgtggaggaacaacaacattgaggatcttactgggtgaattgagctgcttagctatggatcccacagttctacccatcaataagtgcttttgtggtagtcttgtggcttccatatctggggagcttcatttgcctttatagtattaaccttcttgttgttaatcctccacgtacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactgga

SEQ ID NO: 73 - DNA, 4903 bp, Artificial Sequence
  pLF311
cgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttgtcaccttccctgtccttatgcatcttatatctctccacctccaggattttaagccctaggactgggaaggtgacaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcggg

SEQ ID NO: 74 - DNA, 4903 bp, Artificial Sequence
  pLF312
cgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttggggaagaaatggcaagcgacttatgcatcttatatctctccacctccaggattttaagccctagtcgattgccatttcttccccagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcggg

SEQ ID NO: 75 - DNA, 5288 bp, Artificial Sequence
  pLF309
ccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagttggtcgcaagatttaagaattgaggatcttactgggtgaattgagctgcttagctatggatcccacagttctacccatcaataagtgcttttgtggtagtcttgtggcttccatatctggggagcttcatttgcctttatagtattaaccttctttcttaatacttgcgaccaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactgga

SEQ ID NO: 76 - DNA, 4903 bp, Artificial Sequence
  pLF310
cgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttaagggacgaggaggaaggaacttatgcatcttatatctctccacctccaggattttaagccctagttcattcctcctcgtcccttagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcggg

SEQ ID NO: 77 - DNA, 4903 bp, Artificial Sequence
  pLF314
cgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcggg

SEQ ID NO: 78 - DNA, 5487 bp, Artificial Sequence
  pKR2070
cgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacgg

SEQ ID NO: 79 - DNA, 5487 bp, Artificial Sequence
  pKR2071
cgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtttaaacagtcgactctagacttaattaaggatccggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagtaggatcagggactaagtgtttgaggatcttactgggtgaattgagctgcttagctatggatcccacagttctacccatcaataagtgcttttgtggtagtcttgtggcttccatatctggggagcttcatttgcctttatagtattaaccttctacacttaaaccctgatcctacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacgg

SEQ ID NO: 80 - DNA, 5487 bp, Artificial Sequence; identical to SEQ ID NO: 79
  pKR2072

SEQ ID NO: 81 - DNA, 5102 bp, Artificial Sequence
  pKR2073
cgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtttaaacagtcgactctagacttaattaaggatccggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttccctttcttcattcttccctcttatgcatcttatatctctccacctccaggattttaagccctagaggcaagaatgaagaaagggagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacgg

SEQ ID NO: 82 - DNA, 5102 bp, Artificial Sequence
  pKR2075
cgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtttaaacagtcgactctagacttaattaaggatccggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttacacttaaaccctgatcctacttatgcatcttatatctctccacctccaggattttaagccctagtagcatcagggtttaagtgtagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacgg

SEQ ID NO: 83 - DNA, 5102 bp, Artificial Sequence
  pKR2074
cgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtttaaacagtcgactctagacttaattaaggatccggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgcttttttgccgctctcatcgtcttcttatgcatcttatatctctccacctccaggattttaagccctagaagtcgatgagagcggcaaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacgg

SEQ ID NO: 84 - DNA, 958 bp, Artificial Sequence
  amiRNA precursor 159-Atfad2a
ttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 85 - DNA, 958 bp, Artificial Sequence
  amiRNA precursor 159-Atfad2b
ttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 86 - DNA, 958 bp, Artificial Sequence
  amiRNA precursor 159-Atfad2c
ttctagctagctagggtttgggtagtgagtgtaataaagttgcaaagtttttggttaggttacgttttgaccttattattatagttcaaagggaaacattaattaaaggggattatgaagtcgcttgccataacttccccttgaggatcttactgggtgaattgagctgcttagctatggatcccacagttctacccatcaataagtgcttttgtggtagtcttgtggcttccatatctggggagcttcatttgcctttatagtattaaccttctggggaagaaatggcaagcgacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 87 - DNA, 574 bp, Artificial Sequence
  amiRNA precursor 396b-Atfad2a
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttaagggacgaggaggaaggaacttatgcatcttatatctctccacctccaggattttaagccctagttcattcctcctcgtcccttagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 88 - DNA, 574 bp, Artificial Sequence
  amiRNA precursor 396b-Atfad2b
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttgtcaccttccctgtccttatgcatcttatatctctccacctccaggattttaagccctaggactgggaaggtgacaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 89 - DNA, 574 bp, Artificial Sequence
  amiRNA precursor 396b-Atfad2c
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttggggaagaaatggcaagcgacttatgcatcttatatctctccacctccaggattttaagccctagtcgattgccatttcttccccagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 90 - DNA, 958 bp, Artificial Sequence
  amiRNA precursor 159-Atfad3a
ttctagctagctagggtttgggtagtgagtgtaataaagttgcaaagtttttggttaggttacgttttgaccttattattatagttcaaagggaaacattaattaaaggggattatgaagtacgtggaggaacaacaacattgaggatcttactgggtgaattgagctgcttagctatggatcccacagttctacccatcaataagtgcttttgtggtagtcttgtggcttccatatctggggagcttcatttgcctttatagtattaaccttcttgttgttaatcctccacgtacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 91 - DNA, 958 bp, Artificial Sequence
  amiRNA precursor 159-Atfad3b
ttctagctagctagggtttgggtagtgagtgtaataaagttgcaaagtttttggttaggttacgttttgaccttattattatagttcaaagggaaacattaattaaaggggattatgaagttggtcgcaagatttaagaattgaggatcttactgggtgaattgagctgcttagctatggatcccacagttctacccatcaataagtgcttttgtggtagtcttgtggcttccatatctggggagcttcatttgcctttatagtattaaccttctttcttaatacttgcgaccaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 92 - DNA, 574 bp, Artificial Sequence
  amiRNA precursor 396-Atfad3a
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgcttttgttgttaatcctccacgtacttatgcatcttatatctctccacctccaggattttaagccctagtacctggaggattaacaacaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 93 - DNA, 574 bp, Artificial Sequence
  amiRNA precursor 396b-Atfad3b
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 94 - DNA, 958 bp, Artificial Sequence
  amiRNA precursor 159-AtfaeIa
ttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 95 - DNA, 958 bp, Artificial Sequence
  amiRNA precursor 159-AtfaeIb
ttctagctagctagggtttgggtagtgagtgtaataaagttgcaaagtttttggttaggttacgttttgaccttattattatagttcaaagggaaacattaattaaaggggattatgaagtaggatcagggactaagtgtttgaggatcttactgggtgaattgagctgcttagctatggatcccacagttctacccatcaataagtgcttttgtggtagtcttgtggcttccatatctggggagcttcatttgcctttatagtattaaccttctacacttaaaccctgatcctacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 96 - DNA, 958 bp, Artificial Sequence
  amiRNA precursor 159-AtfaeIc
ttctagctagctagggtttgggtagtgagtgtaataaagttgcaaagtttttggttaggttacgttttgaccttattattatagttcaaagggaaacattaattaaaggggattatgaagaagacgatgagtccggcaaattgaggatcttactgggtgaattgagctgcttagctatggatcccacagttctacccatcaataagtgcttttgtggtagtcttgtggcttccatatctggggagcttcatttgcctttatagtattaaccttcttttgccgctctcatcgtcttcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 97 - DNA, 574 bp, Artificial Sequence
  amiRNA precursor 396b-AtfaeIa
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttccctttcttcattcttccctcttatgcatcttatatctctccacctccaggattttaagccctagaggcaagaatgaagaaagggagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 98 - DNA, 574 bp, Artificial Sequence
  amiRNA precursor 396b-AtfaeIb
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttacacttaaaccctgatcctacttatgcatcttatatctctccacctccaggattttaagccctagtagcatcagggtttaagtgtagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 99 - DNA, 574 bp, Artificial Sequence
  amiRNA precursor 396b-AtfaeIc
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgcttttttgccgctctcatcgtcttcttatgcatcttatatctctccacctccaggattttaagccctagaagtcgatgagagcggcaaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 100 - DNA, 958 bp, Artificial Sequence
  Soy genomic miRNA precursor 159
ttctagctagctagggtttgggtagtgagtgtaataaagttgcaaagtttttggttaggttacgttttgaccttattattatagttcaaagggaaacattaattaaaggggattatgaagtggagctccttgaagtccaattgaggatcttactgggtgaattgagctgcttagctatggatcccacagttctacccatcaataagtgcttttgtggtagtcttgtggcttccatatctggggagcttcatttgcctttatagtattaaccttctttggattgaagggagctctacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatct

SEQ ID NO: 101 - DNA, 574 bp, Artificial Sequence
  Soy genomic miRNA precursor 396b
gcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgcttttccacagctttcttgaacttcttatgcatcttatatctctccacctccaggattttaagccctagaagctcaagaaagctgtgggagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 102 - DNA, 1556 bp, Artificial Sequence
  159-fad2a/396b-fad3b amiRNA
ggccgcttgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagc

SEQ ID NO: 103 - DNA, 6077 bp, Artificial Sequence
  PHP55335
ggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 104 - DNA, 1556 bp, Artificial Sequence
  159-fad2b/396b-fad3b amiRNA
ggccgcttgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagc

SEQ ID NO: 105 - DNA, 6077 bp, Artificial Sequence
  PHP55337
ggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 106 - DNA, 1556 bp, Artificial Sequence
  396b-fad3b/159-fad2a amiRNA
ggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagc

SEQ ID NO: 107 - DNA, 6077 bp, Artificial Sequence
  PHP55360
ggcccaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 108 - DNA, 1556 bp, Artificial Sequence
  396b-fad3b/159-fad2b amiRNA
ggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagc

SEQ ID NO: 109 - DNA, 6077 bp, Artificial Sequence
  PHP55361
ggcccaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 110 - DNA, 2530 bp, Artificial Sequence
  159-fad2a/396b-fad3b/159-faeIa amiRNA
ggccgcttgggccgcttgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 111 - DNA, 7051 bp, Artificial Sequence
  PHP55617
ggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcttgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagc

SEQ ID NO: 112 - DNA, 2530 bp, Artificial Sequence
  396b-fad3b/159-fad2a/159faeIa amiRNA
ggccgcttgggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagcggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 113 - DNA, 7051 bp, Artificial Sequence
  PHP55618
ggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagc

SEQ ID NO: 114 - DNA, 2530 bp, Artificial Sequence
  159-fad2b/396b-fad3b/159-faela amiRNA
ggccgcttgggccgcttgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 115 - DNA, 7051 bp, Artificial Sequence
  PHP55635
ggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcttgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagc

SEQ ID NO: 116 - DNA, 2530 bp, Artificial Sequence
  396b-fad3b/159-fad2b/159-FaeIa amiRNA
ggccgcttgggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagcggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgc

SEQ ID NO: 117 - DNA, 7051 bp, Artificial Sequence
  PHP55636
ggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacaagcttggatccgtcgacggcgcgccggatccttaattaagtctagagtcgactgtttaaacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgggggcgcgccaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagc

SEQ ID NO: 118 - DNA, 8394 bp, Artificial Sequence
  PHP56776
ggagatccaagcttggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgagatccgcccatccggccggccagatcctgcaggctagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgcttgggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatttcgcaccaaatcaatgaaagtaataatgaaaagtctgaataagaatacttaggcttagatgcctttgttacttgtgtaaaataacttgagtcatgtacctttggcggaaacagaataaataaaaggtgaaattccaatgctctatgtataagttagtaatacttaatgtgttctacggttgtttcaatatcatcaaactctaattgaaactttagaaccacaaatctcaatcttttcttaatgaaatgaaaaatcttaattgtaccatgtttatgttaaacaccttacaattggttggagaggaggaccaaccgatgggacaacattgggagaaagagattcaatggagatttggataggagaacaacattctttttcacttcaatacaagatgagtgcaacactaaggatatgtatgagactttcagaagctacgacaacatagatgagtgaggtggtgattcctagcaagaaagacattagaggaagccaaaatcgaacaaggaagacatcaagggcaagagacaggaccatccatctcaggaaaaggagctttgggatagtccgagaagttgtacaagaaattttttggagggtgagtgatgcattgctggtgactttaactcaatcaaaattgagaaagaaagaaaagggagggggctcacatgtgaatagaagggaaacgggagaattttacagttttgatctaatgggcatcccagctagtggtaacatattcaccatgtttaaccttcacgtacgtctagaggatcccccgggctgca

SEQ ID NO: 119 - DNA, 8394 bp, Artificial Sequence
  PHP56777
ggagatccaagcttggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgagatccgcccatccggccggccagatcctgcaggctagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgcttgggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatttcgcaccaaatcaatgaaagtaataatgaaaagtctgaataagaatacttaggcttagatgcctttgttacttgtgtaaaataacttgagtcatgtacctttggcggaaacagaataaataaaaggtgaaattccaatgctctatgtataagttagtaatacttaatgtgttctacggttgtttcaatatcatcaaactctaattgaaactttagaaccacaaatctcaatcttttcttaatgaaatgaaaaatcttaattgtaccatgtttatgttaaacaccttacaattggttggagaggaggaccaaccgatgggacaacattgggagaaagagattcaatggagatttggataggagaacaacattctttttcacttcaatacaagatgagtgcaacactaaggatatgtatgagactttcagaagctacgacaacatagatgagtgaggtggtgattcctagcaagaaagacattagaggaagccaaaatcgaacaaggaagacatcaagggcaagagacaggaccatccatctcaggaaaaggagctttgggatagtccgagaagttgtacaagaaattttttggagggtgagtgatgcattgctggtgactttaactcaatcaaaattgagaaagaaagaaaagggagggggctcacatgtgaatagaagggaaacgggagaattttacagttttgatctaatgggcatcccagctagtggtaacatattcaccatgtttaaccttcacgtacgtctagaggatcccccgggctgca

SEQ ID NO: 120 - DNA, 8394 bp, Artificial Sequence
  PHP56802
ggagatccaagcttggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgagatccgcccatccggccggccagatcctgcaggctagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgcttgggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatttcgcaccaaatcaatgaaagtaataatgaaaagtctgaataagaatacttaggcttagatgcctttgttacttgtgtaaaataacttgagtcatgtacctttggcggaaacagaataaataaaaggtgaaattccaatgctctatgtataagttagtaatacttaatgtgttctacggttgtttcaatatcatcaaactctaattgaaactttagaaccacaaatctcaatcttttcttaatgaaatgaaaaatcttaattgtaccatgtttatgttaaacaccttacaattggttggagaggaggaccaaccgatgggacaacattgggagaaagagattcaatggagatttggataggagaacaacattctttttcacttcaatacaagatgagtgcaacactaaggatatgtatgagactttcagaagctacgacaacatagatgagtgaggtggtgattcctagcaagaaagacattagaggaagccaaaatcgaacaaggaagacatcaagggcaagagacaggaccatccatctcaggaaaaggagctttgggatagtccgagaagttgtacaagaaattttttggagggtgagtgatgcattgctggtgactttaactcaatcaaaattgagaaagaaagaaaagggagggggctcacatgtgaatagaagggaaacgggagaattttacagttttgatctaatgggcatcccagctagtggtaacatattcaccatgtttaaccttcacgtacgtctagaggatcccccgggctgca

SEQ ID NO: 121 - DNA, 8394 bp, Artificial Sequence
  PHP56803
ggagatccaagcttggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgggcgcgccgtcgacggatccgtacgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataatgggccgcttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggcccaagcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaaaggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggaacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacgtactttctagacgtacgagatccgcccatccggccggccagatcctgcaggctagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgcttgggcccttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcatttcgcaccaaatcaatgaaagtaataatgaaaagtctgaataagaatacttaggcttagatgcctttgttacttgtgtaaaataacttgagtcatgtacctttggcggaaacagaataaataaaaggtgaaattccaatgctctatgtataagttagtaatacttaatgtgttctacggttgtttcaatatcatcaaactctaattgaaactttagaaccacaaatctcaatcttttcttaatgaaatgaaaaatcttaattgtaccatgtttatgttaaacaccttacaattggttggagaggaggaccaaccgatgggacaacattgggagaaagagattcaatggagatttggataggagaacaacattctttttcacttcaatacaagatgagtgcaacactaaggatatgtatgagactttcagaagctacgacaacatagatgagtgaggtggtgattcctagcaagaaagacattagaggaagccaaaatcgaacaaggaagacatcaagggcaagagacaggaccatccatctcaggaaaaggagctttgggatagtccgagaagttgtacaagaaattttttggagggtgagtgatgcattgctggtgactttaactcaatcaaaattgagaaagaaagaaaagggagggggctcacatgtgaatagaagggaaacgggagaattttacagttttgatctaatgggcatcccagctagtggtaacatattcaccatgtttaaccttcacgtacgtctagaggatcccccgggctgca

SEQ ID NO: 122 - DNA, 1155 bp, Artificial Sequence
  Camelina FAD2-1 HQ008320
atgggtgcaggtggaagaatgccggttccttcttcttcttccaagaaatcagaaaccgatgccataaagcgtgtgccttgcgagaaaccgccgttcacactgggagaattgaagaaagcgatcccaccgcagtgtttcaaacgctctatccctcgctctttctcctaccttatcactgacatcattgttgcctcctgcttctactacgtcgccaccaattacttctctctcctccctcagcctctctcttacttggcttggcccctctattgggcttgtcaaggctgtgtcctaaccggtgtctgggtcatagcccacgaatgcggtcaccacgcattcagcgactaccaatggcttgatgacacagttggtcttatcttccattccttccttctcgtcccttacttctcctggaagtacagtcatcgccgtcaccattccaacacaggatctctcgaaagagatgaagtatttgtcccaaagcagaagtccgctatcaagtggtatggcaaatacctcaacaaccctgctggacgcatcatgatgttaaccgtccagtttgtcctcgggtggcccttgtacttggcctttaacgtctcgggcagaccatacgatgggttcgcttgccatttcttccccaacgctcccatctacaacgaccgtgaacgcctccagatatatctctctgatgccggtattctagcagtctgttttgggctttaccgttacgccgctgcacaaggattggcctcgatgatctgcctctacggagtaccacttctgatagtaaacgcgttcctcgtcttgatcacttacttgcagcacactcatcctgcgttgcctcactacgattcatccgagtgggattggcttaggggagctttggctaccgtagacagagactatggaatcttgaacaaggtgttccacaacatcacggacacacatgtggctcatcatctgttttcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatga

SEQ ID NO: 123 - DNA, 1155 bp, Artificial Sequence
  Camelina FAD2-2 HQ008321
atgggtgcaggtggaagaatgccagttccttcttcttcttccaagaaatctgaaaccgatgccataaagcgtgtgccctgcgagaaaccaccgttcacgctgggagatctgaagaaagcaatcccaccgcagtgtttcaaacgctctatccctcgctctttctcctaccttatcactgacatcattattgcctcctgcttctactacgtcgccaccaattacttttctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacaggatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgtgaacgcctccagatatatctctctgatgccggtattctagcagtctgttttgggctttaccgttacgccgctgcacaaggattggcctcgatgatctgcctctacggagtaccacttctgatagtgaacgcgttcctcgtcttgatcacttacttgcagcacactcatcctgcgttgcctcactacgattcatccgagtgggattggcttaggggagctttggctaccgtagacagagactatggaatcttgaataaggtgttccacaacatcacggacacacatgtggctcatcatctgttctcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatga

SEQ ID NO: 124 - DNA, 1155 bp, Artificial Sequence
  Camelina FAD2-3 HQ008322
atgggtgcaggtggaagaatgccagttccttcttcttcttccaagaaatctgaaaccgatgccataaagcgtgtgccctgcgagaaaccgccgttcacgcttggagaactgaagaaagcaatccccccgcagtgtttcaaacgctctatccctcgctctttctcctaccttatcactgacatcattgttgcctcctgcttctactacgtcgccaccaattacttctctctcctccctcagcctctctcttacttggcttggcctctctactgggcctgtcaaggctgtgtcctaaccggtgtctgggtcatagctcacgaatgcggtcaccacgcattcagcgactaccaatggcttgatgacacagttggtcttatcttccattccttccttctcgtcccttacttctcctggaagtacagtcatcgtcgtcaccattccaacacaggatctctcgaaagagatgaagtatttgtcccaaagcagaaatcagctatcaagtggtatggcaaatacctcaacaaccctcctggacgcatcatgatgttaaccgtccagtttgtcctcgggtggcccttgtacttggcctttaacgtctcgggcagaccgtacgacgggttcgcttgccatttcttccccaacgctcccatctacaacgaccgcgaacgcctccagatatatctctctgatgccggtatcctagcagtctgttttgggctttaccgttacgttgctgcacaaggaatggcctcgatgatctgcctctacggagtaccgcttctgatagtgaacgcgttcctcgtcttgatcacttacttgcagcacactcatcctgcgttgcctcactacgattcatccgagtgggattggcttaggggagctttggctaccgttgacagagactatggaatcttgaacaaggtgttccacaacatcacggacacacatgtggctcatcatctgttctcgacaatgccacattataatgcgatggaagctacaaaggcgataaagccaatactcggtgactactaccagttcgacggaacaccgtggtatgtggcgatgtatagggaggcaaaggagtgtatctatgtagaaccggacagagaaggtgacaagaaaggtgtgtactggtacaacaataagttatga

SEQ ID NO: 125 - DNA, 1164 bp, Artificial Sequence; identical to SEQ ID NO: 35
  Camelina CN101980 FAD3 pLAT13-42rc

SEQ ID NO: 126 - DNA, 1518 bp, Artificial Sequence
  CsFAEI-A GU929420
atgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggtcctaa

SEQ ID NO: 127 - DNA, 1518 bp, Artificial Sequence
  CsFAEI-B GU929421
atgacgtccgttaacgcaaagctcctttaccattacgttctaaccaactttttcaacctttgcttgtttccgttaacggcgttacttgccggaaaagcctctaagcttacagcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacgtacagtccccagggactcattaacgtgcccccacaaaagacctttgcagcttcacgtgaagagacagagcaggtaatcatcggtgcgctagaaaagctattcgagaacaccaaagtaaaccctagagagattggtatacttgtggtgaactcaagcatgtttaatccaactccttcgctatctgcgatggtcgttaacactttcaagctccgaagcaacatcaaaagctttagtctcggaggaatgggttgtagtgctggtgttatcgccattgatcttgcaaaggacttgttgcatgttcataaaaacacttatgcacttgtggtgagcactgagaacatcactcaaggcatttatgctggcgaaaacagatccatgatggttagcaattgcttgtttcgtgttggtggggcagcgattttgctctccaacaaactgggagatcggagacggtccaagtacaagctatgtcatactgttcgaacgcataccggagctgatgacaagtcttttcgatgtgtgcaacaaggagacgatgagggcggtaaaatcggagtttgtctgtcaaaggacataaccgttgttgcggggacagcgcttaagaaaaacatagcaacgttgggtccgttgattcttcctttaagcgaaaagtttctgtttttagttaccttcatcgccaagaaacttttgaaggacaagatcaagcactgttacgtcccggatttcaagcttgctatcgaccatttctgtattcatgcgggaggcagagccgtgatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttcggcgaatagtccttgggaagattgcatcgatagatatccggttcaaattgattctgattcatcaaaatcagagactcatgtcaaaaacggtcggtcctaa

SEQ ID NO: 128 - DNA, 1518 bp, Artificial Sequence
  CsFAEI-C GU929422
atgacgtccgttaacgcaaagctcctttaccattacgtcctaaccaactttttcaacctttgcttgtttccgttaacggcgttacttgccggaaaagcctctacgcttaccacaaacgatctctaccacttctattcccatctccaacacaaccttgtaaccgtaattttactctttgctttctcctctttcggtttggttctctacgttgtaacccggcgcagaccggtttacctcgttgactactcgtgctaccttccaccaccgcatctcaaagttagtgtttctaaggtcatggatattttctaccaaataagaaaagctgatacctcacgaaacgtggcatgcgatgatccatcctcgcttgatttcctgaggaagattcaagaacgttcaggtctaggtgatgaaacctacagtcccccgggactcattcacgtgcccccacaaaagacttttgcagcttcacgtgaagagacagagcaggtaatcatcggtgcgttagaaaagttattcgagaacaccaaagttaaccctagagagattggtatacttgtggtcaactcaagcatgtttaatccaactccttcgctatctgcgatggtcgttaacactttcaagctccgaagcaacatcaaaagctttagtctcggaggaatgggttgtagtgctggtgtcatcgccattgatcttgcaaaggacttgttgcatgttcataaacacacttatgcacttgtggtgagcactgagaacatcactcaaggcatttatgctggcgaaaatagatccatgatggttagcaattgcttgtttcgtgttggtggggcagcgattttgctatccaacaagccgggagatcggagacggtccaagtacaagctatgtcacacggttcggactcataccggagctgatgacaagtcttttcgatgtgtgcaacaaggagacgatgagagcggtaaaatcggagtttgtctgtcaaaggacataacagttgttgcggggacagcgcttaagaaaaacatagcaacgttaggtccgttgattcttcctttaagcgaaaagtttctgttcttagttaccttcatcgccaagaaacttttgaaggacaagatcaagcactattacgtcccggatttcaagcttgctattgaccatttctgtattcatgccggaggcagagccgtaatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttccgcgaatagtccttgggaacattgcatcgatagatatccggttcaaattgattctgattcatcaaaatcagagactcatgtcaaaaacggtcggtcctaa

SEQ ID NO: 129 - DNA, 4599 bp, Artificial Sequence
  FAD2 amiRBA construct
ttctagctagctagggtttgggtagtgagtgtaataaagttgcaaagtttttggttaggttacgttttgaccttattattatagttcaaagggaaacattaattaaaggggattatgaagttccttcctcccagtcccttttgaggatcttactgggtgaattgagctgcttagctatggatcccacagttctacccatcaataagtgcttttgtggtagtcttgtggcttccatatctggggagcttcatttgcctttatagtattaaccttctaagggacgaggaggaaggaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgttctagctagctagggtttgggtagtgagtgtaataaagttgcaaagtttttggttaggttacgttttgaccttattattatagttcaaagggaaacattaattaaaggggattatgaaggacagggaaggacacaagaattgaggatcttactgggtgaattgagctgcttagctatggatcccacagttctacccatcaataagtgcttttgtggtagtcttgtggcttccatatctggggagcttcatttgcctttatagtattaaccttctttcttgtcaccttccctgtccacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgttctagctagctagggtttgggtagtgagtgtaataaagttgcaaagtttttggttaggttacgttttgaccttattattatagttcaaagggaaacattaattaaaggggattatgaagtcgcttgccataacttccccttgaggatcttactgggtgaattgagctgcttagctatggatcccacagttctacccatcaataagtgcttttgtggtagtcttgtggcttccatatctggggagcttcatttgcctttatagtattaaccttctggggaagaaatggcaagcgacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctggcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttaagggacgaggaggaaggaacttatgcatcttatatctctccacctccaggattttaagccctagttcattcctcctcgtcccttagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataaggcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttgtcaccttccctgtccttatgcatcttatatctctccacctccaggattttaagccctaggactgggaaggtgacaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataaggcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttggggaagaaatggcaagcgacttatgcatcttatatctctccacctccaggattttaagccctagtcgattgccatttcttccccagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 130 - DNA, 3064 bp, Artificial Sequence
  FAD3 amiRNA construct
ttctagctagctagggtttgggtagtgagtgtaataaagttgcaaagtttttggttaggttacgttttgaccttattattatagttcaaagggaaacattaattaaaggggattatgaagtacgtggaggaacaacaacattgaggatcttactgggtgaattgagctgcttagctatggatcccacagttctacccatcaataagtgcttttgtggtagtcttgtggcttccatatctggggagcttcatttgcctttatagtattaaccttcttgttgttaatcctccacgtacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgtctagctagctagggtttgggtagtgagtgtaataaagttgcaaagtttttggttaggttacgttttgaccttattattatagttcaaagggaaacattaattaaaggggattatgaagttggtcgcaagatttaagaattgaggatcttactgggtgaattgagctgcttagctatggatcccacagttctacccatcaataagtgcttttgtggtagtcttgtggcttccatatctggggagcttcatttgcctttatagtattaaccttctttcttaatacttgcgaccaacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgcttttgttgttaatcctccacgtacttatgcatcttatatctctccacctccaggattttaagccctagtacctggaggattaacaacaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataaggcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttttcttaatacttgcgaccaacttatgcatcttatatctctccacctccaggattttaagccctagttgctcgcaagtattaagaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 131 - DNA, 4599 bp, Artificial Sequence
  FAE1 amiRNA construct
ttctagctagctagggtttgggtagtgagtgtaataaagttgcaaagtttttggttaggttacgttttgaccttattattatagttcaaagggaaacattaattaaaggggattatgaagagggaagaatgtcgaaagggttgaggatcttactgggtgaattgagctgcttagctatggatcccacagttctacccatcaataagtgcttttgtggtagtcttgtggcttccatatctggggagcttcatttgcctttatagtattaaccttctccctttcttcattcttccctcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgttctagctagctagggtttgggtagtgagtgtaataaagttgcaaagtttttggttaggttacgttttgaccttattattatagttcaaagggaaacattaattaaaggggattatgaagtaggatcagggactaagtgtttgaggatcttactgggtgaattgagctgcttagctatggatcccacagttctacccatcaataagtgcttttgtggtagtcttgtggcttccatatctggggagcttcatttgcctttatagtattaaccttctacacttaaaccctgatcctacacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcagatttgtttataaaaatccaacaatccaaaaaatcagtaagaattatgtacacaaatttagacaagacattaccatattatttcaaacaaaaattatcctgaagagacatcaacacactaatatgtcatctttagtttatttatataaaaccgaatctccgtcttgatctttcaataagcactccttttattagaccaacaaataagcccaagaattgtgacatgtctcatcgaccagtcaataaatatgtagacaaataaccaacacaaggccaagagtaagggcaagagccaagagcatagatctgtataaaaaatgttaggtaagagaatttcctaggaaatatgcactcaattcaacaataaaactaacaaagatgacaataaaagtaatagtgtcaattaaaactaattaaaattgattactgcaacaaaagaactttcctaaaaggtcaaaaaagcaaagttcatttcaacctcaccttcgtgatgcacacaaattaagtatggatctaatcatgcacatacctcggacaaggttactccacagcaagaacgggatcatggcaaaagagaaaaacggaaaaacttcaaaaaaaacatttaaagtttagagtctataacaaatttaaattattatgagagaaaagaagagaagggtgaagacgatgagagcggcaaaagaaggttaatactataaaggcaaatgaagctccccagatatggaagccacaagactaccacaaaagcacttattgatgggtagaactgtgggatccatagctaagcagctcaattcacccagtaagatcctcaatttgccggactcatcgtcttcttcataatcccctttaattaatgtttccctttgaactataataataaggtcaaaacgtaacctaaccaaaaactttgcaactttattacactcactacccaaaccctagctagctagaagcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttccctttcttcattcttccctcttatgcatcttatatctctccacctccaggattttaagccctagaggcaagaatgaagaaagggagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataaggcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgctttacacttaaaccctgatcctacttatgcatcttatatctctccacctccaggattttaagccctagtagcatcagggtttaagtgtagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataaggcgagaaactttgtatgggcatggttatttctcacttctcaccctcctttactttcttatgctaaatcctccttcccctatatctccaccctcaacccctttttctcattataacttttggtgcctagatggtgtgtgtgtgtgcgcgcgagagatctgagctcaattttcctctctcaagtcctggtcatgcttttttgccgctctcatcgtcttcttatgcatcttatatctctccacctccaggattttaagccctagaagtcgatgagagcggcaaaagaatatggcaattcaggcttttaattgctttcatttggtaccatcacttgcaagatttcagagtacaaggtgaacacacacatcttcctcttcatcaattctctagtttcatccttatcttttcattcacggtaactctcactaccctctttcatcttataagttataccgggggtgtgatgttgatgagtgtaaattaaatatatgtgatctctttctctggaaaaattttcagtgtgatatacataataatctcttaatctagagattttatggctttgttatatataag

SEQ ID NO: 132 - DNA, 26 bp, Artificial Sequence
  5′ primer for amplification of a FAD2 gene
cgtcagctccagaatcatgggtgcag

SEQ ID NO: 133 - DNA, 26 bp, Artificial Sequence
  3′ primer for amplification of a FAD2 gene
attatgtgatgtgggaagttagtgtt

SEQ ID NO: 134 - DNA, 25 bp, Artificial Sequence
  5′ primer for amplification of a FAD3 gene
atggttgttgctatggacaaacgta

SEQ ID NO: 135 - DNA, 28 bp, Artificial Sequence
  3′ primer for amplification of a FAD3 gene
tttaattgattttagacttgtcagaagc

SEQ ID NO: 136 - DNA, 22 bp, Artificial Sequence
  5′ primer for amplification of a FAE1 gene
acactagctgagaaatgacgtc

SEQ ID NO: 137 - DNA, 23 bp, Artificial Sequence
  3′ primer for amplification of a FAE1 gene
acatcataaacattatcaaatct

SEQ ID NO: 138 - DNA, 20 bp, Artificial Sequence
  5′ primer for amplification of a region of Csact2 reference gene
tgcagaccgtatgagcaaag

SEQ ID NO: 139 - DNA, 20 bp, Artificial Sequence
  3′ primer for amplification of a Csact2 reference gene
gatccaccgatccagacact

SEQ ID NO: 140 - DNA, 20 bp, Artificial Sequence
  5′ primer for amplification of a region of a Csact7 reference gene
ccaggtatcgctgaccgtat

SEQ ID NO: 141 - DNA, 20 bp, Artificial Sequence
  3′ primer for amplification of a region of a Csact7 reference gene
gattgatcctccgatccaga

SEQ ID NO: 142 - DNA, 20 bp, Artificial Sequence
  5′ primer for amplification of a region of a CsgapC1 reference gene
agagccagtcaagtccctca

SEQ ID NO: 143 - DNA, 20 bp, Artificial Sequence
  3′ primer for amplification of a region of a CsgapC1 gene
gacaagcttgggcttcactc

SEQ ID NO: 144 - DNA, 20 bp, Artificial Sequence
  5′ primer for amplification of a region in a Cstub9 reference gene
aggcgctgagttgactgatt

SEQ ID NO: 145 - DNA, 20 bp, Artificial Sequence
  3′ primer for amplificiation of a region of a Cstub9 reference gene
cctcctcccaaagaatgaca

SEQ ID NO: 146 - DNA, 20 bp, Artificial Sequence
  5′ primer for amplification of a region of a Csubi4 reference gene
cggaagcttctgagcttttc

SEQ ID NO: 147 - DNA, 20 bp, Artificial Sequence
  3′ primer for amplification of a region of a Csubi4 reference gene
cccgcatacggaaacataaa

SEQ ID NO: 148 - DNA, 20 bp, Artificial Sequence
  5′ primer for amplification of a region of a Csubi10 reference gene
acgcttgaggtggagagttc

SEQ ID NO: 149 - DNA, 20 bp, Artificial Sequence
  3′ primer for amplification of a region of a Csubi10 reference gene
tctaccgtcctcgagttgct

SEQ ID NO: 150 - DNA, 20 bp, Artificial Sequence
  5′ primer for amplification of a region of a FAD2 gene
aggtgttccacaacatcacg

SEQ ID NO: 151 - DNA, 21 bp, Artificial Sequence
  3′ primer for amplication of a region of a FAD2 gene
caccgagtattggctttatcg

SEQ ID NO: 152 - DNA, 20 bp, Artificial Sequence
  5′ primer for amplification of a region of a FAD3 gene
aaacgacgagtcatgggttc

SEQ ID NO: 153 - DNA, 20 bp, Artificial Sequence
  3′ primer for amplification of a region of a FAD3 gene
gataagcaagcatggggaga

SEQ ID NO: 154 - DNA, 20 bp, Artificial Sequence
  5′ primer for amplification of a region of a FAE1 gene
ccaactccttcgctatctgc

SEQ ID NO: 155 - DNA, 20 bp, Artificial Sequence
  3′ primer for amplification of a region of a FAE1 gene
caagatcaatggcgatgaca

SEQ ID NO: 156 - DNA, 24 bp, Artificial Sequence
  5′ primer for amplication of a region within a beta conglycinin promotor sequence
tcgtattctcttccgccacctcat

SEQ ID NO: 157 - DNA, 24 bp, Artificial Sequence
  3′ primer for amplification of a region within a beta conglycinin promotor sequence
ccataagccgtcacgattcagatg

(19) United States
(12) Patent Application Publication
(10) Pub. No.: US 2016/0032307 A1
(43) Pub. Date: Feb. 4, 2016

(54) MODIFYING THE FATTY ACID PROFILE OF CAMELINA SATIVA OIL

(71) Applicant: Linnaeus Plant Sciences Inc., Vancouver (CA)

(72) Inventors: Debra M. PUTTICK, Saskatoon (CA); Andrea T. TODD, Saskatoon (CA); Carlene T. SARVAS, Saskatoon (CA); Howard G. DAMUDE, Wilmington, DE (US); Brian MCGONIGLE, Wilmington, DE (US)

(21) Appl. No.: 14/705,160

(22) Filed: May 6, 2015

Related U.S. Application Data
(63) Continuation of application No. 13/748,107, filed on Jan. 23, 2013, now abandoned.
(60) Provisional application No. 61/589,806, filed on Jan. 23, 2012.

(51) Int. Cl. C12N 15/82; C11B 1/10

(52) U.S. Cl. CPC C12N 15/8247; C12N 15/8218; C11B 1/10

(57) ABSTRACT

The present disclosure provides methods and compositions for modifying fatty acids in Camelina sativa oil. Fatty Acid Desaturase 2 (FAD2), Fatty Acid Desaturase 3 (FADS), and/or Fatty Acid Elongase 1 (FAE1) genes regulate fatty acid composition in camelina oil.

### CROSS-REFERENCE TO RELATED APPLICATION

This application is a continuation of U.S. application Ser. No. 13/748,107, filed on Jan. 23, 2013 which claims the benefit of U.S. Provisional Application No. 61/589,806, filed Jan. 23, 2012, both of which are incorporated by reference in their entireties.

### SEQUENCE LISTING

This application contains a Sequence Listing electronically submitted via EFS-Web to the United States Patent and Trademark Office as an ASCII text file entitled “US14-705160 Sequence Listing_ST25.txt” having a size of 442 kilobytes and created on Oct. 10, 2015. The information contained in the Sequence Listing is incorporated by reference herein.

### FIELD

The present disclosure relates to the field of molecular biology and the regulation of fatty acid synthesis in planta. More specifically, the present disclosure provides methods and compositions for modifying fatty acid composition in Camelina sativa oil.

### INTRODUCTION

Camelina sativa (L) Crtz. is an oilseed crop with a relatively short growing season, cold and drought-tolerance, and can grow on marginal land using comparatively little fertilizer. Because of its ability to grow in areas and conditions where major food crops do not grown, Camelina has recently been promoted in Canada and the US for use in bioindustrial applications such as biodiesel, lubricants, and oleochemical feedstocks.

Camelina oil is extracted from seed and typically comprises 25-35% monounsaturated fatty acids and 50-60% polyunsaturated fatty acids (PUFA). The high PUFA content confers low oxidative stability in refined oil, and therefore limits camelina oil in industrial applications. In addition to its low oxidative stability, the presence of more than one double bond leads to undesirable byproducts in processes such as metathesis and ozonolysis.

### SUMMARY

The present application provides methodology, constructs, and the like for modifying fatty acids in Camelina sativa oil.

In one aspect, provided is a method for modifying fatty acid profile in Camelina sativa, comprising suppressing expression of FAD2 and FAD3, relative to a control Camelina sativa plant. In an embodiment, the method further comprises comprising suppressing expression of FAD.

In another aspect, there is provided a method for modifying fatty acid profile in Camelina sativa, comprising suppressing expression of FAD3, relative to a control Camelina sativa plant.

In another aspect, there is provided a method for modifying fatty acid profile in Camelina sativa, comprising suppressing expression of FAD2, relative to a control Camelina sativa plant.

In another aspect, provided is a transgenic Camelina sativa plant having suppressed FAD3, relative to a control Camelina sativa plant. In one embodiment, Camelina oil is extracted from the plant.

In another aspect, provided is a transgenic Camelina sativa plant having suppressed FAD2, relative to a control Camelina sativa plant.

Another aspect provides a transgenic Camelina sativa plant having suppressed FAD2 and FAD3, relative to a control Camelina sativa plant. In one embodiment, Camelina oil is extracted from the seed of the plant.

Another aspect provides a transgenic Camelina sativa plant having suppressed FAD2, FAD3, and FAE1, relative to a control Camelina sativa plant.

In another aspect, provided is an isolated nucleic acid molecule comprising FAD3.

Another aspect provides a construct comprising a nucleic acid sequence that suppresses FAD3. In one embodiment, a plant cell comprises the construct.

In another aspect, there is provided a construct comprising an amiRNA set forth in SEQ ID NO: 41, 44, and 47 (FAD2).

In another aspect, there is provided a construct comprising an amiRNA set forth in SEQ ID NO: 50, 53, and 56 (FAD3).

In another aspect, there is provided a construct comprising an amiRNA set forth in SEQ ID NO: 59 and 62 (FAE1).

In another aspect, there is provided method for producing high oleic camelina oil, comprising (a) suppressing FAD2, FAD3, and FAE1 in Camelina sativa, thereby generating a transgenic Camelina, and (b) extracting oil from said transgenic Camelina seed, wherein said oil is high oleic.

In another aspect, there is provided a method for reducing polyunsaturated fatty acids in camelina oil, comprising (a) suppressing FAD2 and FAD3 in Camelina sativa, thereby generating a transgenic Camelina, and (b) extracting oil from said transgenic Camelina seed, wherein said oil has reduced levels of polyunsaturated fatty acids, relative to oil from a non-transgenic plant.

In another aspect, there is provided high oleic camelina oil, wherein said oil comprises at least 60% oleic acid (% of total fatty acid). In some embodiments, high oleic camelina oil refers to camelina oil having at least about 50-90% oleic acid. For example, high oleic camelina oil may have about 50%, 55%, 60%, 65%, 70%, 75%, 80%, 85%, 90%, or 95% oleic acid.

In another aspect, there is provided low linolenic (18:3Δ9, 12, 15) camelina oil, wherein said oil comprises no more than 10% linolenic acid (% of total fatty acid).

In another aspect, there is provided a method for reducing linolenic acid (18:3Δ9, 12, 15) in Camelina sativa, comprising suppressing FAD3, relative to control a Camelina sativa plant.

In another aspect, there is provided a method for increasing ricinoleic acid and decreasing densipolic acid in Camelina sativa, comprising suppressing FAD2 and FAD3, relative to control a Camelina sativa plant that expresses an oleate hydroxylase. In some embodiments, high ricinoleic oil refers to camelina oil having at least about 15-30% ricinoleic acid. For example, high ricinoleic camelina oil may have about 15%, 20%, 25%, or 30% ricinoleic acid.

In another aspect, there is provided Camelina oil having high oleic acid and gondoic acid, and reduced polyunsaturated fatty acids. In one embodiment, the oil is extracted from a plant suppressing FAD2 and FAD3. In some embodiments, high gondoic camelina oil refers to camelina oil having at least about 20-40% gondoic acid. For example, high gondoic camelina oil may have about 20%, 25%, 30%, 35%, or 40% gondoic acid.

In another aspect, there is provided a transgenic plant comprising an amiRNA set forth in SEQ ID NO: 41, 44, and 47 (FAD2).

In another aspect, there is provided a transgenic plant comprising an amiRNA set forth in SEQ ID NO: 50, 53, and 56 (FAD3).

In another aspect, there is provided a transgenic plant comprising an amiRNA set forth in SEQ ID NO: 59 and 62 (FAE1).

In another aspect, there is a method of using an amiRNA for modifying camelina oil profile. In one embodiment, the amiRNA is set forth in SEQ ID NO: 41, 44, and 47 (FAD2); SEQ ID NO: 50, 53, and 56 (FAD3); and/or SEQ ID NO: 59 and 62 (FAE1).

In another aspect, the application provides oil extracted from transgenic Camelina sativa plant having suppressed FAD2, FAD3, and FAE1, relative to a control Camelina sativa plant.

In another aspect, Applicants provide a method for reducing densipolic acid in Camelina sativa, comprising suppressing FAD3, relative to a control Camelina sativa plant.

In another aspect, provided is a method for modifying fatty acid profile in Camelina sativa, comprising suppressing expression of FAD2 and FAE1, relative to a control Camelina sativa plant. In one embodiment, a transgenic Camelina has suppressed expression of FAD2 and FAE1, relative to a control Camelina sativa plant.

In another aspect, Applicants provide a method for modifying fatty acid profile in Camelina sativa, comprising suppressing expression of FAD3 and FAE1, relative to a control Camelina sativa plant. In one embodiment, a transgenic Camelina has suppressed expression of FAD3 and FAE1, relative to a control Camelina sativa plant.

### BRIEF DESCRIPTION OF THE DRAWINGS

FIG. 1: Schematic map of the T-DNA insert containing amiRNA expression cassette and the selectable marker, DsRed. PsoyBcon: seed specific soy β conglycinin promoter; Tphas: phaseolin terminator sequence; PCMV: constitutive casava vein mosaic virus promoter; Tnos: terminator sequence from Agrobacteria nopaline synthase gene.

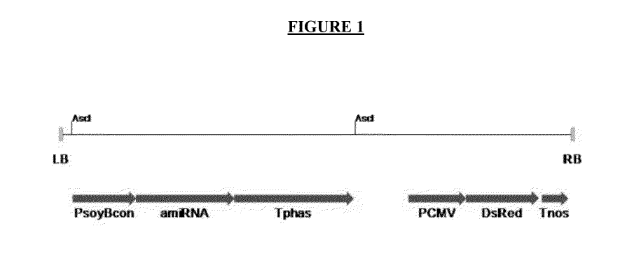

FIG. 2: Target Fatty Acid Profiles of Camelina Seeds.

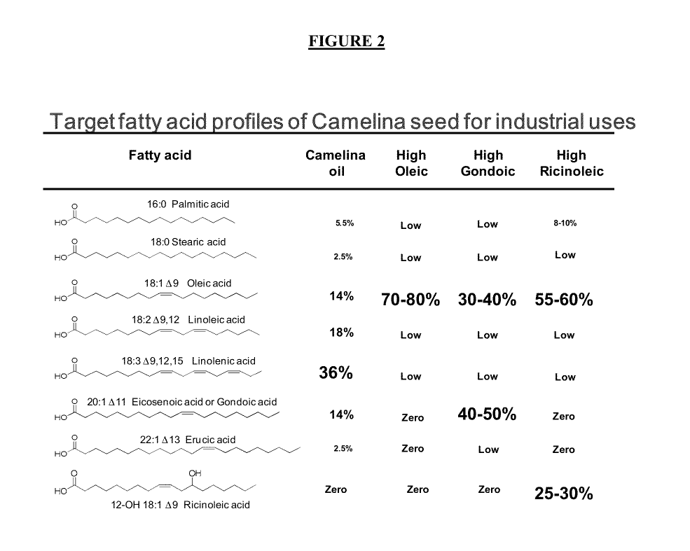

FIG. 3: Fatty Acid Profile of High Oleic Camelina T2 seeds.

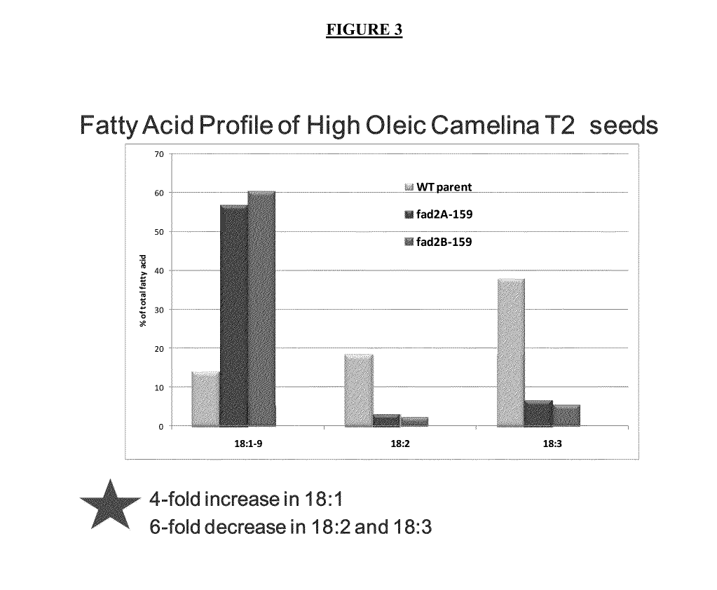

FIG. 4: Fatty Acid Profile of 18:3-Silenced Camelina T2 seeds.

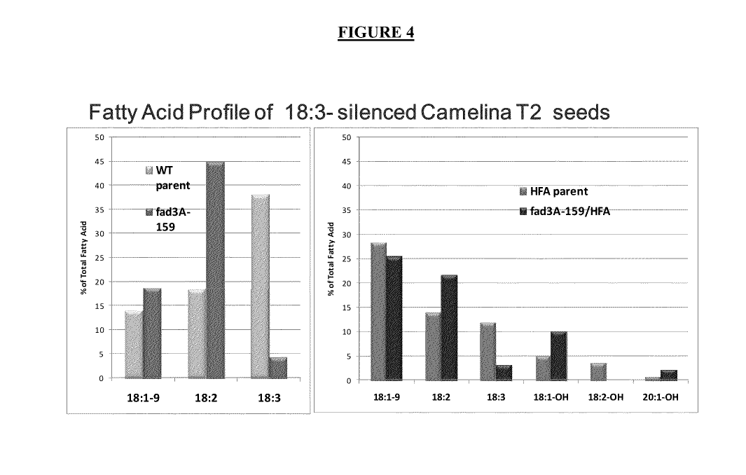

FIG. 5: Schematic map of T-DNA insert of single amiRNA expression cassette. A) FAD2amiRNA and the selectable marker, DsRed; B) FAD3amiRNA and DsRed; and C) FAE1amiRNA and DsRed. PsoyBcon: seed specific soy β conglycinin promoter; Tphas: phaseolin terminator sequence; PCMV: constitutive casava vein mosaic virus promoter; Tnos: terminator sequence from Agrobacteria nopaline synthase gene.

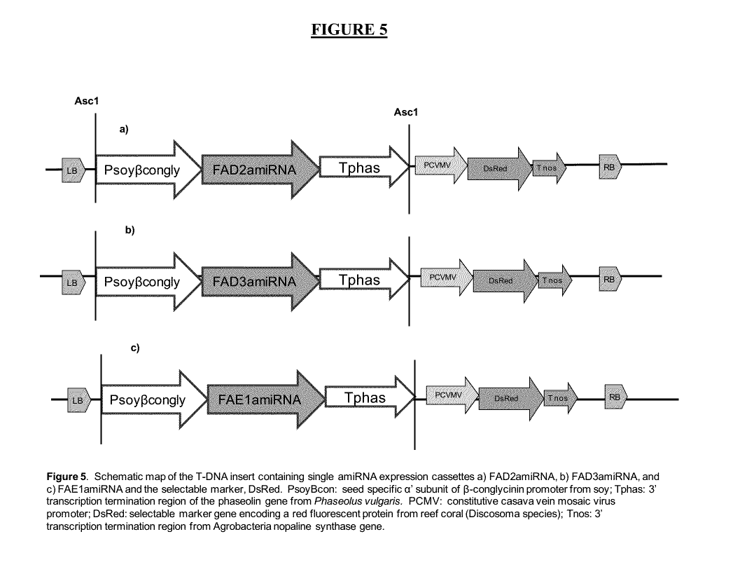

FIG. 6: Schematic map of T-DNA insert of tandem amiRNA expression cassettes. A) Tandem amiRNA expression cassettes FAD2amiRNA, FAD3 amiRNA, and FAE1amiRNA flanked by seed seed specific soy β conglycinin promoter and 3′ transcription termination region of the phaseolin terminator sequence Tphas; B) Tandem amiRNA expression cassettes FAD2amiRNA, FAD3amiRNA, and flanked by seed seed specific soy 3 conglycinin promoter and 3′transcription termination region of the phaseolin terminator sequence Tphas; FAE1 amiRNA expression cassette flanked by the Gy 1 from soy and the 3′ transcription termination region from the pea legumin A2 gene (Rerie et al. (1991) Mol. Gen. Genet. 225: 148-157).

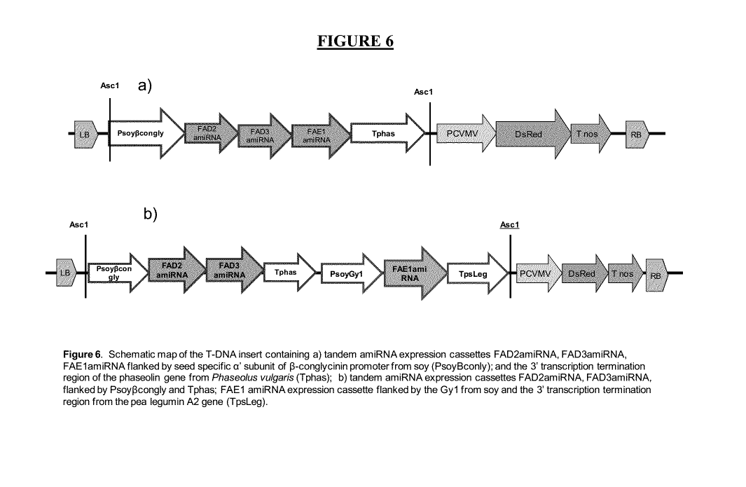

### DETAILED DESCRIPTION

The present inventors recognized that while Camelina sativa may withstand undesirable growth conditions, the fatty acid profile of conventional camelina oil limits its use. Camelina oil typically comprises 25-35% monounsaturated fatty acids and 50-60% polyunsaturated fatty acids (PUFA). The high PUFA content confers low oxidative stability, and therefore limits camelina oil in industrial applications. In addition to its low oxidative stability, the presence of more than one double bond leads to undesirable byproducts in processes such as metathesis and ozono lysis. Thus, in order to compete with other industrial feedstocks, the present inventors contemplated modifying the fatty acid profile of camelina oil.

Therefore, and in one aspect, Applicants contemplate increasing monounsaturated fatty acids such as oleic acid (18:1, cis-9-octadecenoic acid), gondoic acid (20:1, cis-11-icosenoic acid), and erucic acid (22:1, cis-13-docosenoic acid), while decreasing linoleic acid (18:2, cis,cis-9A2-octadecadienoic acid) and alpha linolenic acid (18:3, all-cis-9,12,1-octadecatrienoic acid).

In so doing, Applicants discovered, for example, that the Fatty Acid Desaturase 2 (FAD2), Fatty Acid Desaturase 3 (FAD3), and/or Fatty Acid Elongase 1 (FAE1) genes regulate fatty acid composition in camelina oil. For instance, and in no way limiting, Applicants determined that silencing FAD2, FAD3, and FAE1 produces high oleic camelina oil, while silencing FAD2 and FAD3 reduces PUFA and subsequently increases oleic acid and gondoic acid in Camelina sativa oil.

All technical terms used herein are terms commonly used in biochemistry, molecular biology and agriculture, and can be understood by one of ordinary skill in the art to which this technology belongs. Those technical terms can be found in: MOLECULAR CLONING: A LABORATORY MANUAL, 3rd ed., vol. 1-3, ed. Sambrook and Russel, Cold Spring Harbor Laboratory Press, Cold Spring Harbor, N. Y., 2001; CURRENT PROTOCOLS IN MOLECULAR BIOLOGY, ed. Ausubel et al, Greene Publishing Associates and Wiley-Interscience, New York, 1988 (with periodic updates); SHORT PROTOCOLS IN MOLECULAR BIOLOGY: A COMPENDLUM OF METHODS FROM CURRENT PROTOCOLS IN MOLECULAR BIOLOGY, 5^{TH} ed., vol. 1-2, ed. Ausubel et al, John Wiley & Sons, Inc., 2002; GENOME ANALYSIS: A LABORATORY MANUAL, vol. 1-2, ed. Green et al, Cold Spring Harbor Laboratory Press, Cold Spring Harbor, N. Y., 1997. Methodology involving plant biology techniques is described herein and is described in detail in treatises such as METHODS IN PLANT MOLECULAR BIOLOGY: A LABORATORY COURSE MANUAL, ed. Maliga et al., Cold Spring Harbor Laboratory Press, Cold Spring Harbor, N. Y., 1995. Various techniques using PCR are described, e.g., in Innis et al., PCR PROTOCOLS: A GUIDE TO METHODS AND APPLICATIONS, Academic Press, San Diego, 1990 and in Dieffenbach and Dveksler, PCR PRIMER: A LABORATORY MANUAL, 2^{ND} ed., Cold Spring Harbor Laboratory Press, Cold Spring Harbor, N. Y., 2003. PCR-primer pairs can be derived from known sequences by known techniques such as using computer programs intended for that purpose, e.g., Primer, Version 0.5, 1991, Whitehead Institute for Biomedical Research, Cambridge, Mass. Methods for chemical synthesis of nucleic acids are discussed, for example, in Beaucage and Caruthers, Tetra. Letts. 22:1859-1862 (1981), and Matteucci and Caruthers, J. Am. Chem. Soc. 103:3185 (1981).

Restriction enzyme digestions, phosphorylations, ligations and transformations were done as described in Sambrook et al, MOLECULAR CLONING: A LABORATORY MANUAL, 2^{ND} ed. (1989), Cold Spring Harbor Laboratory Press. All reagents and materials used for the growth and maintenance of bacterial cells were obtained from Aldrich Chemicals (Milwaukee, Wis.), DIFCO Laboratories (Detroit, Mich.), Invitrogen (Gaithersburg, Md.), or Sigma Chemical Company (St. Louis, Mo.) unless otherwise specified.

The terms “encoding” and “coding” refer to the process by which a gene, through the mechanisms of transcription and translation, provides information to a cell from which a series of amino acids can be assembled into a specific amino acid sequence to produce an active enzyme. Because of the degeneracy of the genetic code, certain base changes in a nucleic acid sequence do not change the amino acid sequence of a protein. It is therefore understood that the present disclosure contemplates modifications in any nucleic acid sequence, such that the modification does not alter or affect the function of the encoded protein.

In this description, “expression” denotes the production of the protein product encoded by a gene. “Overexpression” refers to the production of a gene product in transgenic organisms that exceeds levels of production in normal or non-engineered organisms. “Suppression” or “silencing” connotes eliminating or reducing production of the protein product encoded by a gene, relative to a normal, control, non-engineered organism.

“artificial miRNA” or “amiRNA” refers to a small oligoribonucleic acid, typically about 19-25 nucleotides in length, that is not a naturally occurring, and which suppresses expression of a polynucleotide comprising the target sequence transcript or down regulates a target RNA.

Monounsaturated fatty acids include but are not limited to oleic acid (18:1, cis-9-octadecenoic acid), gondoic acid (20:1, cis-11-eicosenoic acid) and erucic acid (22:1, cis-13-docosenoic acid).

Polyunsaturated fatty acids include but are not limited to linoleic acid (18:2, cis,cis-9,12-octadecadienoic acid) and alpha linolenic acid (18:3, all-cis-9,12,15-octadecatrienoic acid).

High oleic camelina oil refers to camelina oil having at least about 50-90% oleic acid. For example, high oleic camelina oil may have about 50%, 55%, 60%, 65%, 70%, 75%, 80%, 85%, 90%, or 95% oleic acid.

High gondoic camelina oil refers to camelina oil having at least about 20-40% gondoic acid. For example, high gondoic camelina oil may have about 20%, 25%, 30%, 35%, or 40% gondoic acid.

High ricinoleic oil refers to camelina oil having at least about 15-30% ricinoleic acid (12-hydroxy-9-cis-octadecenoic acid). For example, high ricinoleic camelina oil may have about 15%, 20%, 25%, or 30% ricinoleic acid.

As used herein, densipolic acid refers to 12-hydroxy-9,15-cis-octadecadienoic acid. In some embodiments, for example, densipolic acid may be reduced by suppressing FAD3 alone.

A. Sequences Affecting Camelina Oil Fatty Acid Profile

The present inventors identified three distinct genes, Fatty Acid Desaturase 2 (FAD2), Fatty Acid Desaturase 3 (FAD3), and Fatty Acid Elongase 1 (FAE1), and determined a role for each in regulating the fatty acid profile of camelina oil. For example, the inventors determined that silencing FAD2 alone produces high oleic camelina oil, while silencing FAD2 and FAD3 together reduces PUFA and subsequently increases oleic acid and gondoic acid in camelina oil.

For purposes of the present disclosure, and non-limiting, an exemplary FAD2 sequence is set forth in any of SEQ ID NOs: 122, 123, and 124, each setting forth an ORE of the published genomic sequence. Likewise, exemplary FAE1 sequences are set forth in SEQ ID NOs. 126, 127, and 128, each setting forth an ORF of the published genomic sequence. These sequences are illustrative because there exists three (3) copies of FAD2 gene (denoted FAD2-1, FAD2-2, and FAD2-3) (Kang, 2011) and FAE1 gene (designated FAE1-A, FAE1-B, and FAE1-C) (Kang, 2011, Hutcheon, 2010) that have approximately 92% identity to each other present in the allohexaploid Camelina genome. FAD3 was heretofore unknown and isolated by the present inventors, and recited in SEQ ID NO: 125.

Of course, the present disclosure contemplates nucleic acid molecules comprised of a variant of any of SEQ ID NOs: 122-128 and 1-101, with one or more bases deleted, substituted, inserted, or added, which variant encodes a polypeptide with activity to similar to that encoded by SEQ ID NOs: 122-128 and 1-101. Accordingly, sequences having “base sequences with one or more bases deleted, substituted, inserted, or added” retain physiological activity even when the encoded amino acid sequence has one or more amino acids substituted, deleted, inserted, or added. For example, the poly A tail or 5′ or 3′ end nontranslation regions may be deleted, and bases may be deleted to the extent that amino acids are deleted. Bases may also be substituted, as long as no frame shift results. Bases also may be “added” to the extent that amino acids are added. It is essential, however, that any such modification does not result in the loss of activity, as normally encoded by SEQ ID NOs: 122-128 and 1-101. A modified nucleic acid in this context can be obtained by modifying the nucleotide base sequences of the invention so that amino acids at specific sites are substituted, deleted, inserted, or added by site-specific mutagenesis, for example, Zoller & Smith, Nucleic Acid Res. 10: 6487-6500 (1982).

For example, and as disclosed herein, SEQ ID NOs: 1, 3, 37, and 39 are pLAT14 clones, each of which is a FAE1 gene. Similarly, SEQ ID NOs: 11, 13, 15, 17, and 19 are pLAT 12 clones, each of which is an FAD2 gene. Likewise, SEQ ID NOs: 21, 23, 25, 27, 29, 31, 33, and 35 are pLAT13 clones, each of which is an FAD3 gene.

As used herein, “sequence identity” or “identity” in the context of two nucleic acid or polypeptide sequences includes reference to the residues in the two sequences which are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of ordinary skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al, Nucleic Acids Res. 16:10881-90 (1988); Huang, et al, Computer Applications in the Biosciences 8: 155-65 (1992), and Pearson, et al., Methods in Molec. Biol. 24:307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol, 215:403-410 (1990); and, Altschul et al., Nucleic Acids Res. 25:3389-3402 (1997).

Software for performing BLAST analyses is publicly available, e.g., through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl Acad Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and X U (Claverie and States, Comput. Chem., 17:191-201 (1993)) low-complexity filters can be employed alone or in combination.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOWS and DIAGONALS SAVED=5.

A sequence affecting fatty acid synthesis and or oil profile may be synthesized ab initio using methods known in the art. For example, a FAD sequence can be synthesized ab initio from the appropriate bases, for example, by using the appropriate protein sequence disclosed here as a guide to create a nucleic acid molecule that, though different from the native nucleic acid sequence, results in a protein with the same or similar amino acid sequence. This type of synthetic nucleic acid molecule is useful when introducing into a plant a nucleic acid sequence, coding for a heterologous protein, that reflects different codon usage frequencies and, if used unmodified, can result in inefficient translation by the host plant.

B. Suppressing Gene Expression

In identifying roles for FAD2, FAD 3, and FAE1, Applicants determined that suppressing FAD2, FAD3, and/or FAE1 confers modified fatty acid oil profile in camelina oil. While any method may be used for suppressing a nucleic acid sequence involved in fatty acid synthesis, the present disclosure contemplates antisense, sense co-suppression, RNAi, artificial microRNA (amiRNA), virus-induced gene silencing (VIGS), antisense, sense co-suppression, and targeted mutagenesis approaches.

RNAi techniques involve stable transformation using RNAi plasmid constructs (Helliwell and Waterhouse, Methods Enzymol. 392:24-35 (2005)). Such plasmids are composed of a fragment of the target gene to be silenced in an inverted repeat structure. The inverted repeats are separated by a spacer, often an intron. The RNAi construct driven by a suitable promoter, for example, the Cauliflower mosaic virus (CaMV) 35S promoter, is integrated into the plant genome and subsequent transcription of the transgene leads to an RNA molecule that folds back on itself to form a double-stranded hairpin RNA. This double-stranded RNA structure is recognized by the plant and cut into small RNAs (about 21 nucleotides long) called small interfering RNAs (siRNAs). siRNAs associate with a protein complex (RISC) which goes on to direct degradation of the mRNA for the target gene.

Artificial microRNA (amiRNA) techniques exploit the microRNA (miRNA) pathway that functions to silence endogenous genes in plants and other eukaryotes (Schwab et al, Plant Cell 18:1121-33 (2006); Alvarez et al, Plant Cell 18:1134-51 (2006)). In this method, 21 nucleotide long fragments of the gene to be silenced are introduced into a pre-miRNA gene to form a pre-amiRNA construct. The pre-miRNA construct is transferred into the plant genome using transformation methods apparent to one skilled in the art. After transcription of the pre-amiRNA, processing yields amiRNAs that target genes, which share nucleotide identity with the 21 nucleotide amiRNA sequence.

In RNAi silencing techniques, two factors can influence the choice of length of the fragment. The shorter the fragment the less frequently effective silencing will be achieved, but very long hairpins increase the chance of recombination in bacterial host strains. The effectiveness of silencing also appears to be gene dependent and could reflect accessibility of target mRNA or the relative abundances of the target mRNA and the hpRNA in cells in which the gene is active. A fragment length of between 100 and 800 bp, preferably between 300 and 600 bp, is generally suitable to maximize the efficiency of silencing obtained. The other consideration is the part of the gene to be targeted. 5′ UTR, coding region, and 3′ UTR fragments can be used with equally good results. As the mechanism of silencing depends on sequence homology there is potential for cross-silencing of related mRNA sequences. Where this is not desirable a region with low sequence similarity to other sequences, such as a 5′ or 3′ UTR, should be chosen. The rule for avoiding cross-homology silencing appears to be to use sequences that do not have blocks of sequence identity of over 20 bases between the construct and the non-target gene sequences. Many of these same principles apply to selection of target regions for designing amiRNAs.

Virus-induced gene silencing (VIGS) techniques are a variation of RNAi techniques that exploits the endogenous-antiviral defenses of plants. Infection of plants with recombinant VIGS viruses containing fragments of host DNA leads to post-transcriptional gene silencing for the target gene. In one embodiment, a tobacco rattle virus (TRV) based VIGS system can be used. Tobacco rattle virus based VIGS systems are described for example, in Baulcombe, Curr. Opin. Plant Biol. 2: 109-113 (1999); Lu, et al, Methods 30: 296-303 (2003); Ratcliff, et al, The Plant Journal 25: 237-245 (2001); and U.S. Pat. No. 7,229,829.

Antisense techniques involve introducing into a plant an antisense oligonucleotide that will bind to the messenger RNA (mRNA) produced by the gene of interest. The “antisense” oligonucleotide has a base sequence complementary to the gene's messenger RNA (mRNA), which is called the “sense” sequence. Activity of the sense segment of the mRNA is blocked by the anti-sense mRNA segment, thereby effectively inactivating gene expression. Application of antisense to gene silencing in plants is described in more detail in Stam et al., Plant J. 21:27-42 (2000).

Sense co-suppression techniques involve introducing a highly expressed sense transgene into a plant resulting in reduced expression of both the transgene and the endogenous gene (Depicker and van Montagu, Curr. Opin. Cell Biol. 9: 373-82 (1997)). The effect depends on sequence identity between transgene and endogenous gene.

Targeted mutagenesis techniques, for example TILLING (Targeting Induced Local Lesions IN Genomes) and “delete-a-gene” using fast-neutron bombardment, may be used to knockout gene function in a plant (Henikoff, et al, Plant Physiol. 135: 630-6 (2004); Li et al, Plant J 27: 235-242 (2001)). TILLING involves treating seeds or individual cells with a mutagen to cause point mutations that are then discovered in genes of interest using a sensitive method for single-nucleotide mutation detection. Detection of desired mutations (e.g. mutations resulting in the inactivation of the gene product of interest) may be accomplished, for example, by PCR methods. For example, oligonucleotide primers derived from the gene of interest may be prepared and PCR may be used to amplify regions of the gene of interest from plants in the mutagenized population. Amplified mutant genes may be annealed to wild-type genes to find mismatches between the mutant genes and wild-type genes. Detected differences may be traced back to the plants which had the mutant gene thereby revealing which mutagenized plants will have the desired expression (e.g. silencing of the gene of interest). These plants may then be selectively bred to produce a population having the desired expression. TILLING can provide an allelic series that includes missense and knockout mutations, which exhibit reduced expression of the targeted gene. TILLING is touted as a possible approach to gene knockout that does not involve introduction of transgenes, and therefore may be more acceptable to consumers. Fast-neutron bombardment induces mutations, i.e. deletions, in plant genomes that can also be detected using PCR in a manner similar to TILLING.

Regardless of the methodology employed, as used here “suppression” or “silencing” or “inhibition” are used interchangeably to denote the down-regulation of the expression of the product of a target sequence relative to its normal expression level in a wild type organism. Suppression includes expression that is decreased by about 10%, 15%, 20%, 25%, 30%, 35%, 40%, 45%, 50%, 55%, 60%, 65%, 70%, 75%, 80%, 85%, 90%, 95%, or 100% relative to the wild type expression level.

C. Nucleic Acid Constructs

The present disclosure comprehends a nucleic acid construct can be used to suppress at least one of FAD2, FAD3, and/or FAE1, and introducing such construct into a plant or cell. Thus, such a nucleic acid construct can be used to suppress at least one of FAD2, FAD3, and/or FAE1 in a plant or cell.

Recombinant nucleic acid constructs may be made using standard techniques. For example, a nucleic acid sequence for transcription may be obtained by treating a vector containing said sequence with restriction enzymes to cut out an appropriate segment. A nucleic acid sequence for transcription may also be generated by annealing and ligating synthetic oligonucleotides or by using synthetic oligonucleotides in a polymerase chain reaction (PCR) to give suitable restriction sites at each end. The nucleic acid sequence is then is cloned into a vector containing suitable regulatory elements, such as upstream promoter and downstream terminator sequences.

Illustrative promoters include constitutive promoters, such as the carnation etched ring virus (CERV), cauliflower mosaic virus (CaMV) 35S promoter, the double enhanced cauliflower mosaic virus promoter, comprising two CaMV 35S promoters in tandem (referred to as a “Double 35S” promoter). Tissue-specific, tissue-preferred, cell type-specific, and inducible promoters may be desirable under certain circumstances. For example, a tissue-specific promoter allows for overexpression or suppression in certain tissues without affecting expression in other tissues. In one embodiment, the present disclosure contemplates a seed-specific promoter, such as the β conglycinin promoter from soybean.

A construct may also contain a termination sequence, positioned downstream of the nucleic acid molecule, such that transcription of mRA is terminated, and polyA sequences added. Exemplary of such terminators include Agrobacterium tumefaciens nopaline synthase terminator (Tnos), Agrobacterium tumefaciens mannopine synthase terminator (Tmas) and the CaMV 35S terminator (T35S). In one embodiment, the present disclosure contemplates a phaseolin terminator. The expression vector also may contain enhancers, start codons, splicing signal sequences, and targeting sequences.

A construct may also comprise a selection marker by which genetically engineered cells can be identified in culture. The marker may be associated with the heterologous nucleic acid molecule, i.e., the gene operably linked to a promoter. For example, and non-limiting, the selectable marker DsRed may be driven by the Cassava Vein Mosaic Virus promoter. As used herein, the term “marker” refers to a gene encoding a trait or a phenotype that permits the selection of, or the screening for, a plant or cell containing the marker. In plants, for example, the marker gene could encode antibiotic or herbicide resistance. This allows for selection of transformed cells from among cells that are not transformed or transfected.

Examples of suitable selectable markers include adenosine deaminase, dihydrofolate reductase, hygromycin-B-phosphotransferase, thymidine kinase, xanthine-guanine phospho-ribosyltransferase, glyphosate and glufosinate resistance, and amino-glycoside 3′-0-phosphotransferase (kanamycin, neomycin and G418 resistance). These markers may include resistance to G418, hygromycin, bleomycin, kanamycin, and gentamicin. The construct may also contain the selectable marker gene bar that confers resistance to herbicidal phosphinothricin analogs like ammonium gluphosinate. Thompson et al, EMBO J. 9: 2519-23 (1987). In one embodiment, a selectable marker comprises DsRed (Clontech Laboratories, I. 2005), driven by the cassava vein mosaic virus promoter. Other suitable selection markers are known as well.

Visible markers such as green florescent protein (GFP) may be used. Methods for identifying or selecting transformed plants based on the control of cell division have also been described. See WO 2000/052168 and WO 2001/059086.

Replication sequences, of bacterial or viral origin, may also be included to allow the vector to be cloned in a bacterial or phage host. Preferably, a broad host range prokaryotic origin of replication is used. A selectable marker for bacteria may be included to allow selection of bacterial cells bearing the desired construct. Suitable prokaryotic selectable markers also include resistance to antibiotics such as kanamycin or tetracycline.

Other nucleic acid sequences encoding additional functions may also be present in the vector, as is known in the art. For instance, when Agrobacterium is the host, T-DNA sequences may be included to facilitate the subsequent transfer to and incorporation into plant chromosomes.

Such gene constructs may suitably be screened for activity by transformation into a host plant via Agrobacterium and screening for modified fatty acid profiles.

Suitably, the nucleotide sequences for the genes may be extracted from the GENBANK® nucleotide database and searched for restriction enzymes that do not cut. These restriction sites may be added to the genes by conventional methods such as incorporating these sites in PCR primers or by sub-cloning.

Preferably, constructs are comprised within a vector, most suitably an expression vector adapted for expression in an appropriate host (plant) cell. Any vector capable of producing a plant comprising the introduced DNA sequence will suffice.

Suitable vectors are well known to those skilled in the art and are described in general technical references such as Pouwels et al, Cloning Vectors. A Laboratory Manual, Elsevier, Amsterdam (1986). Particularly suitable vectors include the Ti plasmid vectors.

D. Plants for Genetic Engineering

The present disclosure comprehends the genetic manipulation of plants, especially Camelina sativa, to suppress FAD2, FAD3, and/or FAE1. The resultant camelina oil has a modified fatty acid profile.

In this description, “plant” denotes any cellulose-containing plant material that can be genetically manipulated, including but not limited to differentiated or undifferentiated plant cells, protoplasts, whole plants, plant tissues, or plant organs, or any component of a plant such as a leaf, stem, root, bud, tuber, fruit, rhizome, or the like.

Other oil-producing plants also are included in this context. Illustrative crops include but are not limited to cotton, soybean, flax, corn, rapeseed, olive, coconut, sunflower, safflower, palm, peanut, castor bean, sesame, various nuts, and citrus.

In the present description, “transgenic plant” refers to a plant that has incorporated a nucleic acid sequence, including but not limited to genes that are not normally present in a host plant genome, nucleic acid sequences not normally transcribed into RNA or translated into a protein (“expressed”), or any other genes or nucleic acid sequences that one desires to introduce into the non-transformed plant, such as genes that normally may be present in the non-transformed plant but that one desires either to genetically engineer or to have altered expression. The “transgenic plant” category includes both a primary transformant and a plant that includes a transformant in its lineage, e.g., by way of standard introgression or another breeding procedure.

It is contemplated that, in some instances, the genome of an inventive transgenic plant will have been augmented through the stable introduction of a transgene. In other instances, however, the introduced gene will replace an endogenous sequence.

E. Genetic Engineering

Exemplary constructs and vectors may be introduced into a host cell using any suitable genetic engineering technique. Both monocotyledonous and dicotyledonous angiosperm or gymnosperm plant cells may be genetically engineered in various ways known to the art. For example, see Klein et al, Biotechnology 4: 583-590 (1993); Bechtold et al, C. R. Acad. Sci. Paris 316:1194-1199 (1993); Bent et al, Mol Gen. Genet. 204:383-396 (1986); Paszowski et al, EMBO J. 3: 2717-2722 (1984); Sagi et al, Plant Cell Rep. 13: 262-266 (1994); and Clough, S. J. and Bent, Plant J., 16(6):735-743 (1998).

For example, and in no way limiting, Agrobacterium species such as A. tumefaciens and A. rhizogenes can be used, for plant transformation. See, for example, Nagel et al, Microbiol Lett. 67: 325 (1990). Briefly, Agrobacterium may be transformed with a plant expression vector via, e.g., electroporation, after which the Agrobacterium is introduced to plant cells via, e.g., the well known leaf-disk method. Additional methods for accomplishing this include but are not limited to electroporation, particle gun bombardment, calcium phosphate precipitation, floral dip, and polyethylene glycol fusion, transfer into germinating pollen grains, direct transformation (Lorz et al, Mol. Genet. 199: 179-182 (1985)), and other methods known to the art. If a selection marker, such as kanamycin resistance, is employed, it makes it easier to determine which cells have been successfully transformed.

The Agrobacterium transformation methods discussed above are known to be useful for transforming dicots. Additionally, de la Pena, et al, Nature 325: 274-276 (1987), Rhodes, et al, Science 240: 204-207 (1988), and Shimamato, et al, Nature 328: 274-276 (1989), all of which are incorporated by reference, have transformed cereal monocots using Agrobacterium. Also see Bechtold, et al, C. R. Acad. Sci. Paris 316 (1994), showing the use of vacuum infiltration for Agrobacterium-mediated transformation.

The presence of a protein, polypeptide, or nucleic acid molecule in a particular cell can be measured to determine if, for example, a cell has been successfully transformed or transfected. The ability to carry out such assay is well known and is not reiterated here.

F. Analyzing Camelina Oil

Transgenic plants of the invention are characterized by modified fatty acid profiles in the seed oil. In some instances, and depending on the target gene(s) suppressed, monounsaturated fatty acids may be increased, while polyunsaturated fatty acids are decreased.

For instance, and in no way limiting, modifying the fatty acid profile of the triacylglycerol in the seed oil of a genetically engineered plant may be achieved by increasing or decreasing the activity of the fatty acid synthesis pathway in the seed, where oil deposition naturally occurs in Camelina.

In describing an illustrative Camelina plant, or extracted camelina oil, “increased monounsaturated fatty acids” refers to a quantitative augmentation in the amount of monounsaturated fatty acids in the plant and/or seed oil when compared to the amount of monounsaturated fatty acids in a wild-type plant and/or seed oil. A quantitative increase in monounsaturated fatty acids can be assayed by several methods, as for example by quantification fatty acid methyl esters by gas chromatography (GC-FAMES). Kunst et al. Plant Physiol Biochem 30:425-434 (1992).

Similarly, an illustrative Camelina plant, or extracted camelina seed oil, may have “decreased polyunsaturated fatty acids,” which refers to a quantitative reduction in the amount of polyunsaturated fatty acids in the plant and/or seed oil when compared to the amount of polyunsaturated fatty acids in a wild-type plant and/or seed oil. A quantitative decrease in polyunsaturated fatty acids can be assayed by several methods, as for example by quantification fatty acid methyl esters by gas chromatography (GC-FAMES). Kunst et al. (1992).

The monounsaturated fatty acids in the instant plants/oil can be increased to levels of about 80% of total seed oil. For example, using the present methodology and constructs, oleic acid was increased to about 60%, and gondoic acid was increased to about 20%.

Likewise, the polyunsaturated fatty acids can be decreased to levels less than 10% of total seed oil.

Specific examples are presented below of methods for obtaining sequences that can modify the fatty acid profile of camelina oil, as well as methods and compositions for introducing such sequences in planta, to produce plant transformants producing oil with a modified fatty acid profile. For example, and as described below and throughout the instant application, Applicants introduced amiRNA sequences based on the endogenous FAD2, FAD3, and FAE1 sequences. The examples are illustrative and non-limiting.

### Example 1

### Isolation of Fatty Acid Desaturases (FAD2 and FAD3) and Fatty Acid Elongase (FAE1)

A. Isolation of FAD2, FAD3, and FAE1 Sequences

RNA was isolated from a pool of green-yellow seed pods from C. sativa line CN101980 as previously described (Meisel et al., 2005) and resuspended in 100 DEPC-treated water. To remove any contaminating genomic DNA, the RNA was mixed with 350 μE RLT lysis buffer, 250 96% ethanol and treated with DNAse I on a Qiagen RNeasy mini column according to the manufacturer's protocol (Qiagen, Hilden). cDNA was made from this RNA using the Superscript II First strand cDNA kit (Invitrogen, Carlsbad) according to the manufacturer's protocol. PCR primers were designed for amplifying the FAD2, FAD3, and FAE1 genes and are provided Table 1 below.

TABLE 1
PCR Primers for Amplification of FAD2,
FAD3 and FAE1 genes.

| Gene | 5′ Primer | 3′ Primer |
|---|---|---|
| FAD2 | CGTCAGCTCCAGAATCATGGGT | ATTATGTGATGTGGGAAGTTAGT |
| | GCAG | GTT |
| | (SEQ ID NO: 132) | (SEQ ID NO: 133) |
| FAD3 | ATGGTTGTTGCTATGGACAAAC | TTTAATTGATTTTAGACTTGTCA |
| | GTA | GAAGCGT |
| | (SEQ ID NO: 134) | (SEQ ID NO ID: 135) |
| FAE1 | ACACTAGCTGAGAAATGACGTC | ACATCATAAACATTATCAAATCT |
| | (SEQ ID NO: 136) | (SEQ ID NO: 137) |

The resulting PCR products were cloned into pCR8/GW/TOPO (Invitrogen, Carlsbad) and the plasmids from at least 16 individual Escherichia coli clones per gene were sent for sequencing. Sequences were aligned using Clustal W2 analysis and these alignments were sent to DuPont to be used for the amiR A design.

B. Gene Expression Analysis

RNA was isolated from five visual stages of seed pod development: 1) flowers, 2) green pods, 3) green-yellow pods, 4) yellow pods, and 5) dried pods as described above. Samples were either pooled from three Camelina plants grown at the same time or were taken from individual T₂ plants. Possible contaminating genomic DNA was removed as described above and cDNA was likewise synthesized.

A sequenced random Camelina EST library was searched using BLAST to find putative reference gene sequences corresponding to the following genes from Arabidopsis thaliana: ACT2 (GenBank U41998), ACT7 (U27811), GAPC1 (NM 111283), TUB9 (M84706), UBI4 (U33014), and UBI10 (NM 178970). Primers for gene expression analysis were designed using Primer3 (Rozen and Skaletsky, 2000) and are provide below in Table 2.

TABLE 2
PCR Primers for Amplification of a Region of
Putative Expression Reference Genes

| Gene | 5′ Primer | 3′ Primer | Product Size (bp) |
|---|---|---|---|
| Csact2 | TGCAGACCGTATGAGCAAAG | GATCCACCGATCCAGACACT | 105 |
| | (SEQ ID NO: 138) | (SEQ ID NO: 139) | |
| Csact7 | CCAGGTATCGCTGACCGTAT | GATTGATCCTCCGATCCAGA | 117 |
| | (SEQ ID NO: 140) | (SEQ ID NO: 141) | |
| CsgapC1 | AGAGCCAGTCAAGTCCCTCA | GACAAGCTTGGGCTTCACTC | 100 |
| | (SEQ ID NO: 142) | (SEQ ID NO: 143) | |
| Cstub9 | AGGCGCTGAGTTGACTGATT | CCTCCTCCCAAAGAATGACA | 102 |
| | (SEQ ID NO: 144) | (SEQ ID NO: 145) | |
| Csubi4 | CGGAAGCTTCTGAGCTTTTC | CCCGCATACGGAAACATAAA | 104 |
| | (SEQ ID NO: 146) | (SEQ ID NO: 147) | |
| Csubi10 | ACGCTTGAGGTGGAGAGTTC | TCTACCGTCCTCGAGTTGCT | 123 |
| | (SEQ ID NO: 148) | (SEQ ID NO: 149) | |

Primers were tested on isolated Camelina genomic DNA and cDNA and all pairs produced a single product.

Interestingly, the Csgapcl and Cstub9 primers produced a larger than predicted product using genomic DNA, likely because they span an intron, which could be useful in determining whether or not there is any contaminating genomic DNA.

The candidate reference genes were tested using the Rotor-Gene SYBR Green Real Time PCR kit (Qiagen, Hilden) on the Rotor-Gene Q machine according to the manufacturer's directions. The templates used were the cDNA samples from the five visual stages of seed pod development and the results for each gene were compared to find the most stable expression levels using geNorm v3.5 (Vandesompele et al., 2002). The genes found to have the most stable expression through all five stages of development were Csgapcl and Csubi4.

Primers for gene expression analysis of the Camelina FAD2, FAD3 and FAE1 genes were designed using Primer 3 (Rozen and Skaletsky, 2000) and are provided below in Table 3.

TABLE 3
PCR Primers for Amplification of a Region of FAD2, FAD3, and/or FAE1

| Gene | 5′ Primer | 3′ Primer | Product Size (bp) |
|---|---|---|---|
| FAD2 | AGGTGTTCCACAACATCACG | CACCGAGTATTGGCTTTATCG | 108 |
| | (SEQ ID NO: 150) | (SEQ ID NO: 151) | |
| FAD3 | AAACGACGAGTCATGGGTTC | GATAAGCAAGCATGGGGAGA | 104 |
| | (SEQ ID NO: 152) | (SEQ ID NO: 153) | |
| FAE1 | CCAACTCCTTCGCTATCTGC | CAAGATCAATGGCGATGACA | 115 |
| | (SEQ ID NO: 154) | (SEQ ID NO: 155) | |

Primers were tested on isolated Camelina genomic DNA and cDNA, and all pairs produced a single product. Interestingly, the FAD3 primers produced a larger than predicted product likely because they span an intron, which could be useful in determining whether or not there is any contaminating genomic DNA.

Gene expression was measured in the five visual stages of seed pod development using the Rotor-Gene SYBR Green Real Time PCR kit (Qiagen, Hilden) on the Rotor-Gene Q machine according to the manufacturer's directions. Each reaction was run in duplicate as a technical replicate and the results were averaged between replicates. The gene expression reactions were analyzed with a melting curve after the PCR run and all reactions gave a single peak melting curve indicative of no contamination. The level of gene expression was calculated relative to the level of expression of the reference gene Csgapcl using the Livak relative method for calculation (Livak and Schmittgen, 2001).

### Example 2

### amiRNA Constructs

Fatty acid biosynthetic gene sequences targeted for silencing by artificial microRNAs (amiRNAs) include FAD2, FAD3, and FAE1 genes. amiRNAs were designed to target both Arabidopsis and Camelina gene families and the corresponding genes targeted along with SEQ ID NOs are provided in Table 4.

TABLE 4
Arabidopsis and Camelina fatty acid biosynthetic
genes targeted for gene silencing

| Organism | | | nt SEQ | nt SEQ |
|---|---|---|---|---|
| Targeted | Gene Family | Gene | ID NO | ID NO |
| Arabidopsis | Fad2 | At3g12120 | 5 | 6 |
| | Fad3 | At2g29980 | 7 | 8 |
| | Fac1 | At4g34520 | 9 | 10 |
| Camelina | Fad2 | pLAT12-1 | 11 | 12 |
| | | pLAT12-4 | 13 | 14 |
| | | pLAT12-11 | 15 | 16 |
| | | pLAT12-12 | 17 | 18 |
| | | pLAT12-13 | 19 | 20 |
| | Fad3 | pLAT13-28 | 21 | 22 |
| | | pLAT13-29 | 23 | 24 |
| | | pLAT13-30 | 25 | 26 |
| | | pLAT13-32 | 27 | 28 |
| | | pLAT13-39 | 29 | 30 |
| | | pLAT13-40 | 31 | 32 |
| | | pLAT13-41 | 33 | 34 |
| | | pLAT13-42rc | 35 | 36 |
| | FaeI | pLAT14-4rci | 37 | 38 |
| | | pLAT14-5rci | 39 | 40 |
| | | pLAT14-7rci | 1 | 2 |
| | | pLAT14-13 | 3 | 4 |

(1) Design of Artificial microRNAs

Artificial microRNAs (amiRNAs) that would have the ability to silence the desired target genes were designed largely according to rules described in Schwab R, et al. (2005) Dev. Cell 8:517-27. To summarize, microRNA sequences are 21 nucleotides in length, start at their 5′-end with a “U”, display 5′ instability relative to their star sequence which is achieved by including a C or G at position 19, and their 10^{th} nucleotide is either an “A” or an “U”. An additional requirement for artificial microRNA design was that the amiRNA have a high free delta-G as calculated using the ZipFold algorithm (Markham, N. R. & Zuker, M. (2005) Nucleic Acids Res. 33: W577-W581.).

### (2) Design of Artificial Star Sequences

“Star sequences” are sequences that base pair with amiRNA sequences, in the precursor RNA, to form imperfect stem structures. To form a perfect stem structure the star sequence would be the exact reverse complement of the amiRNA. The soybean precursor sequence as described in “Novel and nodulation-regulated microRNAs in soybean roots” Subramanian S, Fu Y, Sunkar R, Barbazuk W B, Zhu J, Yu O (2008) BMC Genomics. 9:160 and accessed on mirBase (Conservation and divergence of microRNA families in plants” Dezulian T, Palatnik J F, Huson D H, Weigel D (2005) Genome Biology 6:P13) was folded using mfold (M. Zuker (2003) Nucleic Acids Res. 31: 3406-15; and D. H. Mathews, J. et al. (1999) J. Mol. Biol. 288: 911-940).

The miRNA sequence was then replaced with the amiRNA sequence and the endogenous star sequence was replaced with the exact reverse complement of the amiRNA. Changes in the artificial star sequence were introduced so that the structure of the stem would remain the same as the endogenous structure. The altered sequence was then folded with mfold and the original and altered structures were compared by eye. If necessary, further alternations to the artificial star sequence were introduced to maintain the original structure.

amiRNAs and corresponding STAR sequences that pair with the amiRNAs were designed against the Arabidopsis and Camelina sequences listed in Table 4 using the criteria described above and are listed in Table 5.

TABLE 5
amiRNAs and corresponding STAR sequences targeting Arabidopsis
and Camelina fatty acid biosynthetic sequences

| | | 159 Precursor | 396 Precursor |
|---|---|---|---|
| | amiRNA | STAR sequence | STAR sequence |
| amiRNA | SEQ ID NO: | SEQ ID NO: | SEQ ID NO: |
| fad2a | 41 | 42 | 43 |
| fad2b | 44 | 45 | 46 |
| fad2c | 47 | 48 | 49 |
| fad3a | 50 | 51 | 52 |
| fad3b | 53 | 54 | 55 |
| faeIa | 56 | 57 | 58 |
| faeIb | 59 | 60 | 61 |
| faeIc | 62 | 62 | 63 |

### (3) Conversion of Genomic MicroRNA Precursors to Artificial MicroRNA Precursors

Genomic miRNA precursor genes (“backbones”), such as those described for soy genomic miRNA precursor 159 (SEQ ID NO: 100) or 396b (SEQ ID NO: 101) in US20090155909A1 (WO 2009/079548) and in US20090155910A1 (WO 2009/079532), can be converted to amiRNAs using overlapping PCR, and the resulting DNAs can be completely sequenced and then cloned downstream of an appropriate promoter in a vector capable of transformation.

Alternatively, amiRNAs can be synthesized commercially, for example, by Codon Devices (Cambridge, Mass.), DNA 2.0 (Menlo Park, Calif.) or Genescript (Piscataway, N.J.). The synthesized DNA is then cloned downstream of an appropriate promoter in a vector capable of soybean transformation. Artificial miRNAs can also be constructed using IN-FUSION® technology (Clontech, Mountain View, Calif.).

### (4) Generation of IN-FUSION® Ready Expression Vectors

As described in US20090155910A1, soy genomic miRNA precursor genes were converted to amiRNA precursors 159-fad2-lb and 396b-fad2-lb using overlapping PCR and the resulting precursor amiRNAs were individually cloned downstream of the beta-conglycinin promoter in plasmid PHP27253 (also known as plasmid KS332, described in U.S. Patent Application No. 60/939,872), to form expression constructs PHP32511 and PHP32510, respectively.

The microRNA GM-159 and GM-396b precursors were altered to include Pme I sites immediately flanking the star and microRNA sequences to form the IN-FUSION® ready microRNA precursors. These sequences were cloned into the Not I site of KS332 to form the IN-FUSION® readymicroRNA GM-159-S332 and GM-396b-KS332 plasmids (SEQ ID NO: 65 and 66, respectively).

In order to remove the DSred cassette, GM-396b-KS332 (SEQ ID NO: 66) was digested with BamHI and the fragment containing the GM-396b precursor was re-ligated to produce pKR2007 (SEQ ID NO: 67).

Plasmid GM-159-5332 (SEQ ID NO: 65) was digested with HindIII and the fragment containing the GM-159 precursor was cloned into the HindIII fragment of pKR2007 (SEQ ID NO: 67), containing vector backbone DNA, to produce pKR2009 (SEQ ID NO: 68).

In all of these expression vectors, the expression cassette (beta-conglycinin promoter IN-FUSION® ready microRNA precursor:phaseolin terminator) is flanked by Ascl sites.

(5) Generation of amiRNA Precursors to Silence Arabidopsis and Camelina Fatty Acid Biosynthetic Genes

When synthesizing amiRNA precursors in the GM-159 backbone, the microRNA GM-159 precursor (Example 1) was used as a PCR template. Oligonucleotide pairs were designed for each amiRNA STAR sequence to be amplified using 5′ and 3′ oligonucleotide primers which are identical to the GM-159 precursor region at the 3′ end of the oligonucleotide and which contain either the 21 bp amiRNA or STAR sequence of interest (as listed in Table 5) and a region homologous to either side of the Pmel site of pKR2009 (SEQ ID NO: 68) at the 5′ end of the oligonucleotide. The oligonucleotide primers were designed according to the protocol provided by Clontech and do not leave any footprint of the Pme I sites after the IN-FUSION® recombination reaction.

A similar approach was used to design oligonucleotides for amiRNA precursors in the GM-396b backbone except microRNA GM-396b is used as PCR template and the 5′ region of the oligonucleotide is homologous to either side of the Pmel site of pKR2007 (SEQ ID NO: 68).

The amplified DNA corresponding to each primer set was recombined into either pKR2007 or pKR2009, previously digested with Pmel to linearize the vector, using the manufacturer's protocols provided with the IN-FUSION® kit. In this way, expression vectors for each of the amiRNA/STAR sequences listed in Table 5 were produced.

These plasmids were then digested with Ascl and the fragment containing the amiRNA expression cassette was sub-cloned into the Ascl site of either S 102 (described in WO 02/00904) or pNEB 193 (New England Biolabs). The SEQ ID NOs of sequences for the resulting plasmids containing amiRNA-396b or amiRNA-159 precursors suitable for silencing fad2, fad3 and faeI genes are listed in Table 6.

TABLE 6
(a). amiRNA Expression Constructs For Arabidopsis and Camelina
Fatty Acid Biosynthetic Gene Sequences Targeted for Silencing

| | | amiRNA | | |
|---|---|---|---|---|
| | | Precursor | | nt |
| Gene | amiRNA | SEQ ID | Plasmid | SEQ ID |
| Family | Precursor | NO | Name | NO |
| fad2 | 159-fad2a | 84 | pLF305 | 69 |
| | 159-fad2b | 85 | pLF306 | 70 |
| | 159-fad2c | 86 | pLF308 | 71 |
| | 396b-fad2a | 87 | pLF311 | 72 |
| | 396b-fad2b | 88 | pLF312 | 73 |
| | 396b-fad2c | 89 | pLF313 | 74 |
| fad3 | 159-fad3a | 90 | pLF309 | 75 |
| | 159-fad3b | 91 | pLF310 | 76 |
| | 396b-fad3a | 92 | pLF314 | 77 |
| | 396b-fad3b | 93 | | |
| fael | 159-faeIa | 94 | pKR2070 | 78 |
| | 159-faeIb | 95 | pKR2071 | 79 |
| | 159-faeIc | 96 | pKR2072 | 80 |
| | 396b-faela | 97 | pKR2073 | 81 |
| | 396b-faelb | 98 | pKR2075 | 82 |
| | 396b-faelc | 99 | pKR2074 | 83 |

(b). amiRNA Expression Constructs For Transformed Camelina Lines

| | | | | Trans- |
|---|---|---|---|---|
| | | | | forma- |
| | | Entry | Binary | tion |
| | amiRNA cassette | Vector | Vector | Lines |
| 1 | FAD2A-159 amiRNA | pLF305 | pLAT30a | Dul |
| 2 | FAD2B-159 amiRNA | pLF306 | pLAT31a | Du3 |
| 3 | FAD2C-159 amiRNA | pLF307 | pLAT32b | Du21 |
| 4 | FAD3A-159 amiRNA | pLF308 | pLAT33a | Du5 |
| 5 | FAD3B-159 amiRNA | pLF309 | pLAT34a | DU23 |
| 6 | FAD2A-396b amiRNA | pLF310 | pLAT35a | Du7 |
| 7 | FAD2B-396b amiRNA | pLF311 | pLAT36b | Du25 |
| 8 | FAD2C-396b amiRNA | pLF312 | pLAT37b | Du9 |
| 9 | FAD3A-396b amiRNA | pLF313 | pLAT38b | Du27 |
| 10 | FAD3B-396b amiRNA | pLF314 | pLAT39b | Dull |
| 11 | FAE1A-159 amiRNA | pKR2070 | pLCS3a | Dul3 |
| 12 | FAE1B-159 amiRNA | pKR2071 | pLCS4a | Dul5 |
| 13 | FAE1C-159 amiRNA | pKR2072 | LCS5b | Dul7 |
| 14 | FAE1A-396 amiRNA | pKR2073 | pLCS6b | Dul9 |
| 15 | FAE1C-396 amiRNA | pKR2074 | pLCS7b | Du29 |
| 16 | FAE1B-396 amiRNA | pKR2075 | pLCS8b | Du31 |
| 17 | FAD2A-159/FAD3B-396 | PHP55335 | pLDP42b | Du37 |
| | amiRNA | | | |
| 18 | FAD2B-159/FAD3B-396 | PHP55337 | pLDP43a | Du39 |
| | amiRNA | | | |
| 19 | FAD3B-396/FAD2A-159 | PHP55360 | pLDP44b | Du51 |
| | amiRNA | | | |
| 20 | FAD3B-396/FAD2B-159 | PHP55361 | pLDP45b | Du41 |
| | amiRNA | | | |
| 21 | FAD2A-159/FAD3B-396/ | PHP55617 | pLDP46b | Du43 |
| | FAE1A-159 amiRNA | | | |
| 22 | FAD3B-396/FAD2A-159/ | PHP55618 | pLDP47b | Du45 |
| | FAE1A-159 a miRNA | | | |
| 23 | FAD2B-159/FAD3B-396/ | PHP55635 | pLDP48b | Du47 |
| | FAE1A-159 a miRNA | | | |
| 24 | FAD3B-396/FAD2B-159/ | PHP55636 | pLDP49b | Du49 |
| | FAE1A-159 a miRNA | | | |
| 25 | Pbcon-FAD2A-FAD3B/ | PHP56776 | pLDP52a | Du57 |
| | Pgy-FAElA | | | |
| 26 | Pbcon-FAD2B-FAD3B/ | PHP56777 | pLDP53a | Du59 |
| | Pgy-FAElA | | | |
| 27 | Pbcon-FAD3B-FAD2A/ | PHP56802 | pLDP54a | Du61 |
| | Pgy-FAElA | | | |
| 28 | Pbcon-FAD3B-FAD2B/ | PHP56803 | pLDP55a | Du63 |
| | Pgy-FAElA | | | |

### Table 6(b) Provides Illustrative Constructs and Corresponding Transformed Camelina Lines.

Asc1 fragment from Entry vector was ligated into binary vector containing selectable marker DsRed (Clontech Laboratories, I. 2005). AmiRNA cassettes 1-24 are driven by a single promoter, beta conglycinin from Soybean. Cassettes 25-28 include beta conglycinin promoter for the tandem FAD2/FAD3 amiRNA, and gyl promoter for FAE1 amiRNA. Binary vectors designated a) expression cassette orientation same as selectable marker, b) expression cassette in opposing orientation relative to the selectable marker.

### Example 3

### Camelina Transformation

A. Plant Material

Camelina sativa accession CN101980 was obtained from the Saskatoon Research Station, Agriculture and Agri-Food Canada. Plants were grown in the greenhouse at 22° C. with 16 h light, 8 h dark photoperiod with 20-60% (ambient) humidity and natural lighting enhanced with high pressure sodium lamps.

B. Agrobacterium tumefaciens Strain GV3101pMP90

The recombinant amiRNA vectors described above in Example 2 were introduced to Agrobacterium tumefaciens strain GV3101pMP90 (Koncz and Schell, 1986) by the heat shock method. Transformed colonies were selected on Luria Broth/1.5% agar with 50 mg/L Kanamycin and 25 mg/L Gentamycin.

C. Camelina Transformation

Camelina transformation was performed using a modification of the Arabidopsis floral dip method (Clough, 1998). Briefly, 5 mL cultures of Agrobacterium tumefaciens containing binary vector was grown in Luria broth overnight at 28° C. The 5 mL overnight culture was transferred to a 2 L flask containing 500 mL of the same medium and grown for 16-20 hours at 28° C., 250 rpm shaker incubator. Agrobacteria cells were harvested by centrifugation at 4000 G for 10 minutes and the cell pellets were suspended in 2 L of 5% sucrose containing 0.1% v/v Silwet L77 (Lehle Seeds, Round Rock, Tex., USA).

Camelina was grown in Sunshine Professional Mix, 3 to 4 seeds per 6 inch pot under growth conditions as described above. At the early flowering stage, the floral portion of the Camelina plants were dipped in the Agrobacteria solution as described above. Vacuum infiltration was not required. The treated plants were laid on their sides on absorbent paper and covered with absorbent paper and plastic overnight. In the morning, the plants were uncovered and turned upright. To increase the efficiency of transformation, the process was repeated one week later. The TO plants were then allowed to mature and the T1 seeds were harvested.

D. Screening Camelina T1 Seeds

After harvesting, DsRed-positive T1 seeds were detected by illuminating the seeds under fluorescent light with excitation of 556 nm and 586 emission using a Leica 10446246 filter on a stereoscopic microscope. Plants were self-fertilized and grown to maturity. The T2 seeds were harvested from individual plants for fatty acid analysis.

### Example 4

### Molecular Analysis of Transgenic Camelina Plants

As described above in Example 2, DsRed-positive seeds were germinated and the resulting T 1 plants were confirmed by PCR under standard Taq DNA polymerase conditions (Qiagen) using primers specific for the β conglycinin promoter sequence. Specifically, a forward primer and a reverse primer were designed to amplify a 473 bp region within β conglycinin promoter, common to all of the instant constructs.

| (SEQ ID NO: 156) | |
|---|---|
| Forward primer: | TCGTATTCTCTTCCGCCACCTCAT |
| (SEQ ID NO: 157) | |
| Reverse primer: | CCATAAGCCGTCACGATTCAGATG |

T2 lines were selected that were positive for the β conglycinin promoter sequence.

Selected T2 lines were further characterized by Southern blot analysis. Briefly, genomic DNA was extracted from young leaves using a modification of the Dellaporta DNA extraction method for Maize (Coldspring Harbour Laboratory Manual, 1984). Five micrograms of genomic DNA was digested for 16 hours with Pstl, then electrophoreised on 0.8% agarose gel in 1% TAE buffer for 6 hours at 40 volts. The DNA was transferred onto Amersham Hybond N+ by downward capillary blotting with 0.5 M NaOH and 1.5M NaCl. DNA probe was the β conglycinin promoter, made by PCR amplification as described above.

### Example 5

### Gas Chromatography of Fatty Acid Methyl Esters (GC-FAMES)

To determine total seed fatty acid composition, a pool of 20-30 seeds collected from each individual T1 Camelina plant were placed in Pyrex screw-cap tubes with 2 mL 1 M HCl in methanol (Supelco) and 0.5 mL of hexane. The tubes were tightly capped and heated at 80° C. for 6-16 h. After cooling, 2 mL of 0.9%>NaCl and 1 mL of hexane was added, and fatty acid methyl esters (FAMES) were recovered by collecting the hexane phase. Gas chromatography of FAMES was conducted using an Agilent 6890N GC fitted with a DB-23 capillary column (0.25 mm 30 m, 0.25 uM thickness; J & W, Folsom, Calif., USA) as described previously (Kunst, 1992).

### Example 6

### amiRNA Silencing of FAD2 Genes in Camelina Seeds

For each FAD2 amiRNA construct, at least 20 T1 plants were grown to maturity and the seeds harvested. As shown in Table 7 below, the FAD2B-159 amiRNA construct produced the best silencing of the FAD2 genes resulting in a 9-fold decrease in linoleic acid and 6-fold decrease in t-linolenic acid, and enabling a 4-fold increase in the oleic acid content of Camelina sativa seed oil in pooled seeds of T2 generation. Data represent 20-30 pooled seeds of the best T2 line from each construct.

TABLE 7
Fatty acid compositions of camelina seed oil in FAD2 knockout lines

| | Fatty acid (% area) | | | | | | | | |
|---|---|---|---|---|---|---|---|---|---|
| Line | 16:0 | 18:0 | 18:1-9 | 18:1-11 | 18:2-9, 12 | 18:2-9, 15 | 18:3-9, 12, 15 | 20:0 | 20:1-11 |
| FAD2A-159 | 5.1 | 3.7 | 56.8 | 1.2 | 3.2 | 0.6 | 6.7 | 1.6 | 17.1 |
| FAD2A-396b | 5.7 | 3.6 | 44.3 | 1.1 | 8.2 | | 12.9 | 1.6 | 17.4 |
| FAD2B-159 | 4.8 | 3.1 | 60.4 | 1.0 | 2.2 | 1.7 | 5.4 | 1.2 | 16.7 |
| FAD2B-396b | 4.9 | 3.1 | 54.2 | 1.0 | 2.3 | 1.9 | 8.5 | 1.2 | 18.4 |
| FAD2C-159 | 4.7 | 2.4 | 56.5 | 0.9 | 2.2 | 2.0 | 7.2 | 0.9 | 19.3 |
| FAD2C-396b | 5.3 | 2.6 | 51.5 | 1.0 | 3.0 | 1.7 | 9.2 | 1.21 | 9.5 |
| Null (CN101980) | 6.7 | 2.8 | 15.3 | 1.0 | 19.0 | | 32.4 | 1.8 | 12.6 |

Interestingly, 20:1 also increased up to 1.5-fold in the FAD2 silenced lines. Single seed GC-FAMES of the best lines has shown oleic acid levels as high as 63% in multiple copy lines, and consistently as high as 61% in single copy lines. Single insert lines were grown for further study and homozygous plants were obtained in the T3 generation, as determined by DsRed expression, Southern blot, and qPCR.

Additionally, 18:2-9, 15 was produced in the FAD2-amiRNA transgenic seeds. This fatty acid is not normally present in wild type Camelina, but was earlier discovered and published in Arabidopsis that was transformed with FAD3 (Puttick et al, 2009).

Table 8 below show GC-FAMES data from 23 single seeds of FAD2B-159amiRNA T2 line Du3-27, confirming segregation with 5 null and 18 seeds showing relative 18:1 content between 57 and 61% and corresponding decreased 18:2 and 18:3.

TABLE 8
Fatty acid compositions of FAD2B-159 amiRNA line Du03-27 single seeds
Fatty acid (% area)

| | Fatty acid (% area) | | | | | | | | | |
|---|---|---|---|---|---|---|---|---|---|---|
| FAD2B-159 T2 line | 16:0 | 18:0 | 18:1-9 | 18:1-11 | 18:2-9, 12 | 18:2-9, 15 | 18:3 | 20:0 | 20:1-11 | 22:1 |
| DU03-27-10 | 6.7 | 3.3 | 17.0 | 1.0 | 20.6 | | 28.7 | 2.0 | 13.1 | 2.8 |
| DU03-27-17 | 6.8 | 3.1 | 17.9 | 1.6 | 17.7 | | 26.7 | 1.5 | 10.6 | 2.3 |
| Du03-27-22 | 6.7 | 3.3 | 18.4 | 1.0 | 21.1 | | 26.9 | 2.0 | 13.2 | 2.9 |
| Du03-27-01 | 6.6 | 3.0 | 18.5 | 1.1 | 20.0 | | 29.4 | 1.7 | 12.3 | 2.6 |
| Du03-27-03 | 6.4 | 3.3 | 19.3 | 1.0 | 20.5 | | 27.3 | 2.1 | 13.1 | 2.7 |
| Du03-27-19 | 5.9 | 5.5 | 57.0 | 1.2 | 2.8 | 1.0 | 5.8 | 1.5 | 16.6 | 1.7 |
| Du03-27-20 | 5.6 | 3.2 | 57.6 | 1.4 | 2.5 | 1.4 | 6.4 | 1.1 | 17.8 | 1.8 |
| Du03-27-12 | 5.4 | 2.9 | 58.0 | 1.3 | 2.7 | 1.5 | 6.6 | 1.1 | 17.5 | 1.8 |
| Du03-27-16 | 5.1 | 3.2 | 58.3 | 1.2 | 2.2 | 1.8 | 5.6 | 1.3 | 18.3 | 1.9 |
| Du03-27-13 | 5.2 | 3.4 | 59.0 | 1.2 | 2.6 | 1.3 | 5.9 | 1.3 | 17.6 | 1.6 |
| DU03-27-07 | 5.0 | 3.5 | 59.0 | 1.1 | 2.6 | 1.2 | 5.6 | 1.3 | 17.3 | 1.7 |
| DU03-27-06 | 5.2 | 2.9 | 59.1 | 1.1 | 2.6 | 1.3 | 6.6 | 1.1 | 17.2 | 1.6 |
| Du03-27-05 | 4.9 | 3.0 | 59.4 | 1.1 | 2.4 | 1.5 | 5.9 | 1.2 | 17.3 | 1.6 |
| Du03-27-18 | 5.2 | 3.8 | 59.4 | 1.1 | 1.8 | 1.8 | 5.3 | 1.0 | 17.8 | 1.7 |
| Du03-27-02 | 5.0 | 3.0 | 59.7 | 1.1 | 2.4 | 1.5 | 6.0 | 1.2 | 17.3 | 1.6 |
| Du03-27-09 | 4.7 | 2.7 | 60.0 | 1.1 | 1.8 | 1.6 | 5.5 | 1.0 | 18.3 | 1.7 |
| Du03-27-23 | 4.8 | 2.8 | 60.3 | 1.2 | 2.1 | 1.4 | 5.7 | 1.0 | 17.4 | 1.7 |
| Du03-27-15 | 5.1 | 3.0 | 60.4 | 1.2 | 2.2 | 1.7 | 5.4 | 1.0 | 17.7 | 1.6 |
| Du03-27-08 | 4.9 | 3.0 | 60.5 | 1.1 | 2.3 | 1.6 | 5.8 | 1.2 | 17.0 | 1.5 |
| Du03-27-14 | 5.1 | 3.1 | 60.8 | 1.2 | 1.9 | 1.9 | 5.0 | 1.1 | 17.7 | 1.6 |
| DU03-27-11 | 4.7 | 2.9 | 60.9 | 1.0 | 2.0 | 1.6 | 5.3 | 1.2 | 17.3 | 1.5 |
| DU03-27-21 | 4.9 | 2.8 | 61.0 | 1.3 | 2.0 | 1.5 | 5.5 | 1.1 | 17.2 | 1.6 |
| Du03-27-04 | 4.5 | 2.7 | 61.0 | 0.9 | 1.5 | 2.3 | 4.5 | 1.0 | 18.4 | 1.7 |

### Example 7

### amiRNA Knockout of FAD3 Genes in Camelina Seeds

For each FAD3 amiRNA construct, at least 20 T1 plants were grown to maturity and the seeds harvested. Table 9 below shows the fatty acid profile of camelina seed oil in FAD3 knockout lines. As shown below, silencing FAD3 resulted in significant increases in 18:2 and decreases in 18:3.

TABLE 9
Fatty acid compositions of camelina seed oil in FAD3 knockout lines

| | Fatty acid (% area) | | | | | | | |
|---|---|---|---|---|---|---|---|---|
| Line | 16:0 | 18:0 | 18:1-9 | 18:1-11 | 18:2-9, 12 | 18:3-9, 12, 15 | 20:0 | 20:1-11 |
| FAD3A-159 | 6.7 | 3.0 | 18.7 | 0.9 | 44.8 | 4.3 | 1.8 | 12.3 |
| FAD3A-396b | 6.9 | 2.9 | 11.9 | 0.9 | 32.0 | 18.7 | 2.4 | 13.3 |
| FAD3B-159 | 6.3 | 2.6 | 18.2 | 0.9 | 46.4 | 2.6 | 1.4 | 13.7 |
| FAD3B-396b | 6.3 | 2.3 | 14.2 | 1.0 | 48.7 | 2.7 | 2.0 | 12.4 |
| Null (CN101980) | 6.7 | 2.8 | 15.3 | 1.0 | 19.0 | 32.4 | 1.8 | 12.6 |

### Example 8

### amiRNA Knockout of FAE Genes in Camelina Seeds

For each FAE1 amiRNA construct, at least 20 T1 plants were grown to maturity and the seeds harvested. Table 10 below shows the fatty acid profile of camelina seed oil in FAE1 knockout lines. As shown below, silencing FAE1 increased 18:1-9 (oleic acid) and decreased 20:0 and 20:1-11 (gondoic acid).

TABLE 10
Fatty acid compositions of camelina seed oil in FAEl knockout lines

| | Fatty acid (% area) | | | | | | | |
|---|---|---|---|---|---|---|---|---|
| Line | 16:0 | 18:0 | 18:1-9 | 18:1-11 | 18:2-9, 12 | 18:3-9, 12, 15 | 20:0 | 20:1-11 |
| FAE1A-159 | 8.5 | 2.8 | 22.7 | 1.4 | 22.5 | 39.2 | 0.9 | 0.5 |
| FAE1A-396b | 7.6 | 3.0 | 22.2 | 1.3 | 19.7 | 41.1 | 0.8 | 2.5 |
| FAE1B-159 | 8.0 | 3.7 | 20.3 | 1.4 | 21.9 | 41.1 | 0.5 | 0.9 |
| FAE1B-396b | 7.6 | 3.2 | 20.9 | 1.2 | 21.6 | 41.1 | 0.5 | 2.0 |
| FAE1C-159 | 7.2 | 3.7 | 19.7 | 1.3 | 24.8 | 36.5 | 0.7 | 3.0 |
| FAE1C-396b | 7.7 | 2.9 | 18.0 | 1.3 | 21.8 | 42.0 | 0.6 | 3.2 |
| Null (CN101980) | 6.9 | 2.9 | 138 | 0.9 | 18.4 | 33.2 | 1.7 | 13.1 |

### Example 9

### Design of Stacked Expression Constructs Suppressing FAD2, FAD3 and FAE1 Genes in Camelina Seeds

Stacked FAD2/FAD3 and FAD2/FAD3/FAE1 amiRNA constructs were made using methods well-known in the art.

For example, using cloning methods familiar to one skilled in the art (e.g. PCR, restriction enzyme digestion, etc.), individual amiRNA precursors targeting either fad2 and fad3, shown in Table 6, were combined together into single transcriptional unit such that both amiRNA precursors were expressed together downstream of the single beta-conglycinin promoter. In some cases, a third amiRNA precursor targeting fae1 was also combined with the fad2 and fad3 amiRNA precursors to generate triple amiRNA units targeting all three genes. In each case, the full cassette including the beta-conglycinin promoter, the multiple amiRNA and the phaseolin transcription terminator were flanked by Ascl sites to enable cloning into other expression vectors.

As shown in Table 6, amiRNA precursors 159-fad2a (SEQ ID NO: 84), 159-fad2b (SEQ ID NO: 85), 159-Fad2c (SEQ ID NO: 86), 159-fad3a (SEQ ID NO: 90), 159-fad3b (SEQ ID NO: 91), 159-faeIa (SEQ ID NO: 94), 159-faeIb (SEQ ID NO: 95) and 159-faeIc (SEQ ID NO: 96) are 958 nt in length and are substantially similar to the deoxyribonucleotide sequence set forth in SEQ ID NO: 100 wherein nucleotides 276 to 296 of SEQ ID NO: 100 are replaced by the fad2a amiRNA (SEQ ID NO: 41), fad2b amiRNA (SEQ ID NO: 44), fad2c amiRNA (SEQ ID NO: 47), fad3a amiRNA (SEQ ID NO: 50), fad3b amiRNA (SEQ ID NO: 53), faela amiRNA (SEQ ID NO: 56), faelb amiRNA (SEQ ID NO: 59) or faelc amiRNA (SEQ ID NO: 62), respectively. Nucleotides 121 to 141 of SEQ ID NO: 100 are replaced by 159-fad2a Star Sequence (SEQ ID NO: 42), 159-fad2b Star Sequence (SEQ ID NO: 45), 159-fad2c Star Sequence (SEQ ID NO: 48), 159-fad3a Star Sequence (SEQ ID NO: 51), 159-FAD3B Star Sequence (SEQ ID NO: 54), 159-faela Star Sequence (SEQ ID NO: 57), 159-faelb Star Sequence (SEQ ID NO: 60) or 159-faelc Star Sequence (SEQ ID NO: 63), respectively.

From Table 6, the amiRNA precursors 396b-fad2a (SEQ ID NO: 87), 396b-fad2b (SEQ ID NO: 88), 396b-fad2c (SEQ ID NO: 89), 396b-fad3a (SEQ ID NO: 92), 396b-fad2b (SEQ ID NO: 93), 396b-faela (SEQ ID NO: 97), 396b-faelb (SEQ ID NO: 98) and 396b-faelc (SEQ ID NO: 99) are 574 nt in length and are substantially similar to the deoxyribonucleotide sequence set forth in SEQ ID NO: 101 wherein nucleotides 196 to 216 of SEQ ID NO: 101 are replaced by the fad2a amiRNA (SEQ ID NO: 41), fad2b amiRNA (SEQ ID NO: 44), fad2c amiRNA (SEQ ID NO: 47), fad3a amiRNA (SEQ ID NO: 50), fad3b amiRNA (SEQ ID NO: 53), faela amiRNA (SEQ ID NO: 56), faelb amiRNA (SEQ ID NO: 59) or faelc amiRNA (SEQ ID NO: 62), respectively and wherein nucleotides 262 to 282 of SEQ ID NO: 101 are replaced by the 396b-fad2a Star Sequence (SEQ ID NO: 43), 396b-fad2b Star Sequence (SEQ ID NO: 46), 396b-fad2c Star Sequence (SEQ ID NO: 49), 396b-fad3a Star Sequence (SEQ ID NO: 52), 396b-fad3b Star Sequence (SEQ ID NO: 55), 396b-faela Star Sequence (SEQ ID NO: 58), 396b-faelb Star Sequence (SEQ ID NO: 61) or 396b-faelc Star Sequence (SEQ ID NO: 64), respectively.

Illustrative amiRNA combinations made and the corresponding vector sequences are described in Table 11.

TABLE 11
Precursor amiRNA combinations and amiRNA Expression
Vectors for Camelina Fatty Acid Biosynthetic
Gene Sequences Targeted for Silencing.

| | amiRNA | | |
|---|---|---|---|
| amiRNA | Precursor | Plasmid | Plasmid |
| Precursor | SEQ ID NO | Name | SEQ ID NO |
| 159-fad2a/396b-fad3b amiRNA | 102 | PHP55335 | 103 |
| 159-fad2b/396b- fad3b amiRNA | 104 | PHP55337 | 105 |
| 396b-fad3b/159-fad2a amiRNA | 106 | PHP55360 | 107 |
| 396b-fad3b/159-fad2b amiRNA | 108 | PHP55361 | 109 |
| 159-fad2a/396b-fae1a amiRNA | 110 | PHP55617 | 111 |
| 396b-fad3b/159-fae1a amiRNA | 112 | PHP55618 | 113 |
| 159-fad2b/396b-fae1a amiRNA | 114 | PHP55635 | 115 |
| 396b-fad3b/159-fae1a amiRNA | 116 | PHP55636 | 117 |

As shown in Table 11 above, the amiRNA precursor 159-fad2a/396b-fad3b (SEQ ID NO: 102), which combines amiRNA precursors 159-fad2a (SEQ ID NO: 84) and 396b-fad3b (SEQ ID NO: 91) into one transcriptional unit, is 1556 nt in length and is substantially similar to the deoxyribonucleotide sequence set forth in SEQ ID NO: 100 (from nt 16 to 974 of 159-fad2a/396b-fad3b) wherein nucleotides 276 to 296 of SEQ ID NO: 100 are replaced by 159-fad2a amiRNA (SEQ ID NO: 41) and wherein nucleotides 121 to 141 of SEQ ID NO: 100 are replaced by 159-fad2a amiRNA Star Sequence (SEQ ID NO: 42). The amiRNA precursor 159-fad2a/396b-fad3b (SEQ ID NO: 102) is also substantially similar to the deoxyribonucleotide sequence set forth in SEQ ID NO: 101 (from nt 982 to 1555 of 159-fad2a/396b-fad3b) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55).

From Table 11, the amiRNA precursor 159-fad2b/396b-fad3b (SEQ ID NO: 104), which combines amiRNA precursors 159-fad2b (SEQ ID NO: 85) and 396b-fad3b (SEQ ID NO: 91) into one transcriptional unit, is 1556 nt in length and is substantially similar to the deoxyribonucleotide sequence set forth in SEQ ID NO: 100 (from nt 16 to 974 of 159-fad2b/396b-fad3b) wherein nucleotides 276 to 296 of SEQ ID NO: 100 are replaced by 159-fad2b amiRNA (SEQ ID NO: 44) and wherein nucleotides 121 to 141 of SEQ ID NO: 100 are replaced by 159-fad2b amiRNA Star Sequence (SEQ ID NO: 45). The amiRNA precursor 159-fad2b/396b-fad3b (SEQ ID NO: 104) is also substantially similar to the deoxyribonucleotide sequence set forth in SEQ ID NO: 101 (from nt 982 to 1555 of 159-fad2b/396b-fad3b) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55).

The amiRNA precursor 396b-fad3b/159-fad2a (SEQ ID NO: 106), which combines amiRNA precursors and 396b-fad3b (SEQ ID NO: 91) and 159-fad2a (SEQ ID NO: 84) into one transcriptional unit, is 1556 nt in length and is substantially similar to the deoxyribonucleotide sequence set forth in SEQ ID NO: 101 (from nt 7 to 581 of 396b-fad3b/159-fad2a) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55). The amiRNA precursor 396b-fad3b/159-fad2a (SEQ ID NO: 106) is also substantially similar to the deoxyribonucleotide sequence set forth in SEQ ID NO: 100 (from nt 588 to 1546 of 396b-fad3b/159-fad2a) wherein nucleotides 276 to 296 of SEQ ID NO: 100 are replaced by 159-fad2a amiRNA (SEQ ID NO: 41) and wherein nucleotides 121 to 141 of SEQ ID NO: 100 are replaced by 159-fad2a amiRNA Star Sequence (SEQ ID NO: 42).

The amiRNA precursor 396b-fad3b/159-fad2b (SEQ ID NO: 108), which combines amiRNA precursors and 396b-fad3b (SEQ ID NO: 91) and 159-fad2b (SEQ ID NO: 85) into one transcriptional unit, is 1556 nt in length and is substantially similar to the deoxyribonucleotide sequence set forth in SEQ ID NO: 101 (from nt 7 to 581 of 396b-fad3b/159-fad2b) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55). The amiRNA precursor 396b-fad3b/159-fad2b (SEQ ID NO: 108) is also substantially similar to the deoxyribonucleotide sequence set forth in SEQ ID NO: 100 (from nt 588 to 1546 of 396b-fad3b/159-fad2b) wherein nucleotides 276 to 296 of SEQ ID NO: 100 are replaced by 159-fad2b amiRNA (SEQ ID NO: 44) and wherein nucleotides 121 to 141 of SEQ ID NO: 100 are replaced by 159-fad2b amiRNA Star Sequence (SEQ ID NO: 45).

The amiRNA precursor 159-fad2a/396b-fad3b/159-faela (SEQ ID NO: 110), which combines amiRNA precursors 159-fad2a (SEQ ID NO: 84), 396b-fad3b (SEQ ID NO: 91) and 159-faela (SEQ ID NO: 94) into one transcriptional unit, is 2530 nt in length and is substantially similar to the deoxyribonucleotide sequence set forth in SEQ ID NO: 100 (from nt 25 to 983 of 159-fad2a/396b-fad3b/159-faela) wherein nucleotides 276 to 296 of SEQ ID NO: 100 are replaced by 159-fad2a amiRNA (SEQ ID NO: 41) and wherein nucleotides 121 to 141 of SEQ ID NO: 100 are replaced by 159-fad2a amiRNA Star Sequence (SEQ ID NO: 42). The amiRNA precursor 159-fad2a/396b-fad3b/159-faela (SEQ ID NO: 110) is also substantially similar to the deoxyribonucleotide sequence set forth in SEQ ID NO: 101 (from nt 991 to 1564 of 159-fad2a/396b-fad3b/159-faela) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55). The amiRNA precursor 159-fad2a/396b-fad3b/159-faela (SEQ ID NO: 110) is also substantially similar to the deoxyribonucleotide sequence set forth in SEQ ID NO: 100 (from nt 1571 to 2529 of 159-fad2a/396b-fad3b/159-faela) wherein nucleotides 276 to 296 of SEQ ID NO: 100 are replaced by 159-faela amiRNA (SEQ ID NO: 56) and wherein nucleotides 121 to 141 of SEQ ID NO: 100 are replaced by 159-faela amiRNA Star Sequence (SEQ ID NO: 57).

The amiRNA precursor 396b-fad3b/159-fad2a/159-faela (SEQ ID NO: 112), which combines amiRNA precursors and 396b-fad3b (SEQ ID NO: 91), 159-fad2a (SEQ ID NO: 84) and 159-faela (SEQ ID NO: 94) into one transcriptional unit, is 2530 nt in length and is substantially similar to the deoxyribonucleotide sequence set forth in SEQ ID NO: 101 (from nt 16 to 590 of 396b-fad3b/159-fad2a/159-faela) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55). The amiRNA precursor 396b-fad3b/159-fad2a/159-faela (SEQ ID NO: 112) is also substantially similar to the deoxyribonucleotide sequence set forth in SEQ ID NO: 100 (from nt 597 to 1555 of 396b-fad3b/159-fad2a/159-faela) wherein nucleotides 276 to 296 of SEQ ID NO: 100 are replaced by 159-fad2a amiRNA (SEQ ID NO: 41) and wherein nucleotides 121 to 141 of SEQ ID NO: 100 are replaced by 159-fad2a amiRNA Star Sequence (SEQ ID NO: 42). The amiRNA precursor 396b-fad3b/159-fad2a/159-faela (SEQ ID NO: 112) is also substantially similar to the deoxyribonucleotide sequence set forth in SEQ ID NO: 100 (from nt 1571 to 2529 of 396b-fad3b/159-fad2a/159-faela) wherein nucleotides 276 to 296 of SEQ ID NO: 100 are replaced by 159-faela amiRNA (SEQ ID NO: 56) and wherein nucleotides 121 to 141 of SEQ ID NO: 100 are replaced by 159-faela amiRNA Star Sequence (SEQ ID NO: 57).

From Table 11, the amiRNA precursor 159-fad2b/396b-fad3b/1 59-faela (SEQ ID NO: 114), which combines amiRNA precursors 159-fad2b (SEQ ID NO: 85), 396b-fad3b (SEQ ID NO: 91) and 159-faela (SEQ ID NO: 94) into one transcriptional unit, is 2530 nt in length and is substantially similar to the deoxyribonucleotide sequence set forth in SEQ ID NO: 100 (from nt 25 to 983 of 159-fad2b/396b-fad3b/159-faela) wherein nucleotides 276 to 296 of SEQ ID NO: 100 are replaced by 159-fad2b amiRNA (SEQ ID NO: 44) and wherein nucleotides 121 to 141 of SEQ ID NO: 100 are replaced by 159-fad2a amiRNA Star Sequence (SEQ ID NO: 45). The amiRNA precursor 159-fad2b/396b-fad3b/159-faela (SEQ ID NO: 114) is also substantially similar to the deoxyribonucleotide sequence set forth in SEQ ID NO: 101 (from nt 991 to 1564 of 159-fad2b/396b-fad3b/159-faela) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55). The amiRNA precursor 159-fad2b/396b-fad3b/159-faela (SEQ ID NO: 114) is also substantially similar to the deoxyribonucleotide sequence set forth in SEQ ID NO: 100 (from nt 1571 to 2529 of 159-fad2b/396b-fad3b/159-faela) wherein nucleotides 276 to 296 of SEQ ID NO: 100 are replaced by 159-faela amiRNA (SEQ ID NO: 56) and wherein nucleotides 121 to 141 of SEQ ID NO: 100 are replaced by 159-faela amiRNA Star Sequence (SEQ ID NO: 57).

The amiRNA precursor 396b-fad3b/159-fad2b/159-faela (SEQ ID NO: 116), which combines amiRNA precursors and 396b-fad3b (SEQ ID NO: 91), 159-fad2b (SEQ ID NO: 85) and 159-faela (SEQ ID NO: 94) into one transcriptional unit, is 2530 nt in length and is substantially similar to the deoxyribonucleotide sequence set forth in SEQ ID NO: 101 (from nt 16 to 590 of 396b-fad3b/159-fad2b/159-faela) wherein nucleotides 196 to 216 of SEQ ID NO: 101 are replaced by 396b-fad3b amiRNA (SEQ ID NO: 53) and wherein nucleotides 262 to 282 of SEQ ID NO: 101 are replaced by 396b-fad3b amiRNA Star Sequence (SEQ ID NO: 55). The amiRNA precursor 396b-fad3b/159-fad2b/159-faela (SEQ ID NO: 116) is also substantially similar to the deoxyribonucleotide sequence set forth in SEQ ID NO: 100 (from nt 597 to 1555 of 396b-fad3b/159-fad2b/159-faela) wherein nucleotides 276 to 296 of SEQ ID NO: 100 are replaced by 1 9-fad2b amiRNA (SEQ ID NO: 44) and wherein nucleotides 121 to 141 of SEQ ID NO: 100 are replaced by 159-fad2b amiRNA Star Sequence (SEQ ID NO: 45). The amiRNA precursor 396b-fad3b/159-fad2b/159-faela (SEQ ID NO: 116) is also substantially similar to the deoxyribonucleotide sequence set forth in SEQ ID NO: 100 (from nt 1571 to 2529 of 396b-fad3b/159-fad2b/159-faela) wherein nucleotides 276 to 296 of SEQ ID NO: 100 are replaced by 159-faela amiRNA (SEQ ID NO: 56) and wherein nucleotides 121 to 141 of SEQ ID NO: 100 are replaced by 159-faea amiRNA Star Sequence (SEQ ID NO: 57).

In addition to double amiRNA precursors targeting fad2 and fad3 and triple amiRNA precursors targeting fad2, fad3 and fael, constructs were made where a double amiRNA precursor targeting fad2 and fad3 was placed downstream of the beta-conglycinin promoter and a second amiRNA precursor targeting fael was placed in a separate cassette downstream of the soy glycinin Gyl promoter (Nielsen, N C et al. (1989) Plant Cell 1:313-328). Exemplary amiRNA combinations made and the corresponding vector sequences are described in Table 12.

TABLE 12
Precursor amiRNA combinations and amiRNA Expression Vectors
For Camelina Fatty Acid Biosynthetic Gene Sequences Targeted
for Silencing amiRNA amiRNA amiRNA amiRNA Plasmid Plasmid

| Precur- | Precur- | | Precur- | | |
|---|---|---|---|---|---|
| sor 1 | sor 1 | Precur- | sor 2 | | Plasmid |
| (β-conglycinin | SEQ ID | sor 2 | SEQ ID | Plasmid | SEQ ID |
| promoter) | NO | glycinin Gy1 | NO | Name | NO |
| 159-fad2a/ | 102 | 159-faela | 94 | PHP56776 | 118 |
| 396b-amiRNA | | amiRNA | | | |
| 159-fad2b/ | 104 | 159-faela | 94 | PHP56777 | 119 |
| 396b-fad3b | | amiRNA | | | |
| amiRNA | | | | | |
| 396b-fad3b/ | 106 | 159-faela | 94 | PHP56802 | 120 |
| 159-fad2a | | amiRNA | | | |
| amiRNA | | | | | |
| 396b-fad3b/ | 108 | 159-faela | 94 | PHP56803 | 121 |
| 159 fad2b | | amiRNA | | | |
| amiRNA | | | | | |

### Example 10

### amiRNA Knockout of FAD2, FAD3 and FAE Genes in Camelina Seeds

For each stacked FAD2/FAD3 and FAD2/FAD3/FAE1 amiRNA construct, as disclosed in Example 9 above, at least 20 T1 plants were grown to maturity and the seeds harvested. Table 13 and Table 14 below show the fatty acid profile of camelina seed oil in the stacked knockout lines. In the best lines, silencing FAD2, FAD3, and FAE1 increased 18:1-9 (oleic acid) greater than 6-fold and decreased PUFA (linoleic, linolenic) to less than 10% of total fatty acids, and other MUFA (gondoic, and erucic acid) to less than 4% of total fatty acids in the seed oil.

TABLE 13
Fatty acid compositions of camelina seed oil in knockout lines of stacked
FAD2, FAD3, and FAEl amiRNA tandem driven by Soybean promoter Beta
conglycinin.

| | Fatty acid (% area) | | | | | | |
|---|---|---|---|---|---|---|---|
| Line | 16:0 | 18:0 | 18:1-9 | 18:2-9, 12 | 18:3-9, 12, 15 | 20:0 | 20:1-11 |
| FAD2A-159/FAD3B-396 amiRNA | 4.2 | 2.5 | 50.9 | 14.0 | 3.9 | 18.3 | 1.9 |
| FAD2A-159/FAD3B-396/FAE1A-159 | 6.1 | 2.7 | 66.0 | 10.9 | 5.1 | 4.5 | 0.4 |
| amiRNA | | | | | | | |
| FAD2B-159/FAD3B-396 amiRNA | 5.4 | 3.0 | 58.6 | 5.8 | 3.7 | 16.8 | 1.8 |
| FAD2B-159/FAD3B-396/FAE1A-159 | 4.8 | 1.8 | 79.0 | 4.8 | 3.7 | 2.9 | 0.2 |
| amiRNA | | | | | | | |
| FAD3B-396/FAD2A-159 amiRNA | 5.6 | 2.7 | 45.0 | 20.2 | 2.6 | 16.5 | 2.2 |
| FAD3B-396/FAD2A-159/FAE1A-159 | 6.1 | 2.3 | 57.9 | 20.1 | 2.7 | 6.4 | 0.5 |
| amiRNA | | | | | | | |
| FAD3B-396/FAD2B-159 amiRNA | 5.8 | 3.4 | 52.2 | 11.4 | 3.0 | 17.1 | 2.1 |
| FAD3B-396/FAD2B-159/FAE1A-159 | 5.9 | 2.5 | 60.0 | 13.6 | 5.1 | 7.9 | 0.7 |
| amiRNA | | | | | | | |
| Null (CN101980) | 6.7 | 3.2 | 12.4 | 17.8 | 34.8 | 13.0 | 3.2 |

TABLE 14
Fatty acid compositions of camelina seed oil in knockout lines of stacked
FAD2 and FAD3 amiRNA tandem driven by Soybean promoter Beta conglycinin and
FAEl amiRNA under Soybean Gyl promoter.

| | Fatty acid (% area) | | | | | | |
|---|---|---|---|---|---|---|---|
| Line | 16:0 | 18:0 | 18:1-9 | 18:2-9, 12 | 18:3-9, 12, 15 | 20:0 | 20:1-11 |
| Pbcon-FAD2A-159/FAD3B-396/Pgy- | 6.0 | 3.0 | 74.7 | 7.1 | 3.3 | 3.0 | 0.2 |
| FAElA-159 amiRNA | | | | | | | |
| Pbcon-FAD2B-159/FAD3B-396/Pgy- | 5.6 | 2.8 | 73.7 | 5.1 | 4.2 | 4.2 | 0.3 |
| FAElA-159 amiRNA | | | | | | | |
| Pbcon-FAD3B-396/FAD2B-159/Pgy- | 6.0 | 2.6 | 73.7 | 8.2 | 4.2 | 1.6 | 0.3 |
| FAElA-159 amiRNA | | | | | | | |
| Null (CN101980) | 6.8 | 3.2 | 13.8 | 18.6 | 32.5 | 12.4 | 3.2 |

1. A method for modifying fatty acid profile in Camelina sativa, comprising suppressing expression of FAD2 and/or FAD3, relative to a control Camelina sativa plant.

2. The method of claim 1, further comprising suppressing expression of FAE1.

3. (canceled)

4. A transgenic Camelina sativa plant having suppressed FAD2 and/or FAD3, relative to a control Camelina sativa plant.

5. (canceled)

6. Camelina oil extracted from the plant of claim 4.

7-8. (canceled)

9. A transgenic Camelina sativa plant of claim 4 further having suppressed FAE1 relative to a control Camelina sativa plant.

10. (canceled)

11. A construct comprising a nucleic acid sequence that suppresses FAD3.

12. A plant cell comprising the construct of claim 11.

13. (canceled)

14. A construct of claim 11 comprising an amiRNA set forth in SEQ ID NO: 50, 53, 56 (FAD3).

15-23. (canceled)

24. A transgenic plant of claim 4 comprising an FAD2 amiRNA set forth in SEQ ID NOs: 41, 44, and 47 (FAD2).

25. A transgenic plant of claim 4 comprising an FAD3 amiRNA set forth in SEQ ID NOs: 50, 53, 56 (FAD3).

26. A transgenic plant of claim 4 comprising a FAE1 amiRNA set forth in SEQ ID NOs: 59, 62 (FAE1).

27-28. (canceled)

29. Camelina oil extracted from the plant of claim 9.

30-32. (canceled)
